(19) United States
(12) Patent Application Publication
(10) Pub. No.: US 2024/0189364 A1
(43) Pub. Date: Jun. 13, 2024

(54) VILLI STROMAL CELLS COMPOSITIONS AND USES THEREOF

(71) Applicant: ARUGULA SCIENCES LLC, Farmers Branch, TX (US)

(72) Inventor: Ramon CORONADO, San Antonio, TX (US)

(21) Appl. No.: 18/278,646

(22) PCT Filed: Feb. 28, 2022
(86) PCT No.: PCT/US2022/018108

§ 371 Date: Aug. 24, 2023

Related U.S. Application Data
(60) Provisional application No. 63/154,027, filed on Feb. 26, 2021.

(51) Int. Cl. A61K 35/35; A61P 9/10; A61P 11/00; A61P 29/00; A61P 37/06

(52) U.S. Cl. CPC A61K 35/35; A61P 9/10; A61P 11/00; A61P 29/00; A61P 37/06

(57) ABSTRACT

The invention provides compositions of perinatal stromal cells and methods of use thereof. Specifically, the invention provides composition comprising amniotic stromal cells, Wharton's jelly stromal cells, placenta proper stromal cell, chorionic stromal cells, or chorionic villi stromal cells; and methods of uses thereof for the treatment of graft versus host disease, for reducing fibrosis, for reducing inflammation, for inducing immune tolerance, and for treating single- or multi-organ fibrosis.

### RELATED APPLICATIONS

This application claims priority to U.S. Provisional Application No. 63/154,027 filed on Feb. 26, 2021, entitled “VILLI STROMAL CELLS COMPOSITIONS AND USES THEREOF”, the contents of which are incorporated by reference in their entirety herein.

### BACKGROUND OF THE INVENTION

### Field of the Invention

The present invention relates generally to perinatal stromal cells compositions and more specifically to compositions of chorionic villi-derived stromal cells and uses thereof to treat tissue fibrosis, and tissue inflammation.

### Background Information

Rejection after transplantation continues to be a medical obstacle associated with high morbidity and mortality. Transplantation rejection can be classified as acute (short term) or chronic (long term). Acute rejection is less common and can be managed due to advances in broad spectrum immune-suppressants. On the other hand, chronic rejection has no adequate treatment; representing an important unmet clinical need. In the case of hematopoietic stem cell transplantation, rejection is characterized when donor cells recognize the recipient's cells as “non-self” and engage in a broad attack against host tissues in a process known as graft-versus-host disease (GVHD). Chronic graft-versus-host-disease (cGVHD) continues to be a major immunological complication that develops in 30-60% of transplanted patients. Clinical cGVHD has characteristics of acute rejection but also has a more diverse components that resembles autoimmune syndromes, such as progressive allograft injury, primarily characterized by obliterative arteriopathy and interstitial fibrosis. For example, cGVHD of the lungs could manifests as bronchiolitis obliterans accompanied with airflow obstruction. Similarly, in the liver, hepatic cGVHD could be associated with ductopenia and fibrosis. Evidently, cGVHD shares many of the pathologies associated with organ allograft rejection. Therefore, immunoregulatory molecules are of great interests when developing new drugs to treat cGVHD.

Mesenchymal stromal cells (MSCs) have therapeutic and regenerative applications because of their combination of multipotency, migratory ability, and presumed immunoprivileged state. In addition, MSCs are responsive to the environmental cues encountered after migration to sites of injury. Although it was initially believed that benefits derived from delivering donor stem cells was due to their localization in injured tissue and differentiation into healthy cells, resulting in improved organ repair, the levels of engraftment and survival are less than 5%, a number believed to be too small to be therapeutically relevant. In addition, one of the beneficial effects of MSCs (either bone marrow, BM, or adipose derived, ASC) is related to modulation of the local cell/tissue environment by paracrine signaling. Recent evidence suggest that MSCs (BM and adipose derived) isolated from older individuals and aged animals lose their regenerative capacity with increasing age in part due to changes in their molecular profile. Thus, there is less likelihood that MSCs from old donors will be as efficacious as MSCs from young donors. Many important questions revolve around the loss or gain of factors that are associated with the impaired regenerative capacity of older MSCs.

Perinatal Stromal Cells (PSCs) are cells derived from tissues surrounding the baby after birth. PSCs have been gaining interest in the cell therapy field with the intention of leveraging some of its innate immune-regulation functions during normal pregnancy (i.e., immune tolerance, anti-inflammation etc.). In addition, PSCs can be collected in large numbers from ethically sourced material that would be discarded otherwise. Similar to Mesenchymal Stromal Cells (MSCs), PSCs have been studied for their immunoregulatory properties and capabilities to modulate organ fibrosis in vivo. While bone marrow derived mesenchymal stromal sells have been extensively studied in their ability to regulate GVHD, little information is available for PSCs. Moreover, no information for PSCs is available in a humanized GVHD model. In addition, there are no reports comparing PSCs from different tissue sources in vivo, leaving a gap on understanding how tissue sources could influence their potential therapeutic effect.

Wharton's jelly (WJ) MSCs, as well as whole chorion derived (CSCs) and chorionic villi-derived MSCs (CVCs), an abundant source of “younger” MSCs (i.e., PSCs), may afford several advantages compared to those MSCs isolated from adult tissues. These include unlimited availability of the tissue source, non-invasive isolation, and increased isolation efficiency leading to large numbers of MSCs. In addition, current literature reports that WJ-MSCs display several beneficial features of embryonic stem cells and lack factors associated with tumorigenesis and invasion as previously reported.

### SUMMARY OF THE INVENTION

The present invention is based on the seminal discovery that perinatal stromal tissue cells isolated from amnion, placenta, Wharton's jelly, chorionic membrane, chorionic villi, and umbilical cord tissue are useful for the treatment and/or prevention of graft versus host disease, and specifically for the treatment and/or prevention of tissue fibrosis, such as idiopathic pulmonary fibrosis and multi-organ fibrosis.

In one embodiment, the present invention provides a perinatal stromal cell (PSC) composition comprising at least one of the following cell types: (i) amnion perinatal stromal cells (APSCs), (ii) placenta proper stromal cells (PPSCs), (iii) Wharton's jelly perinatal stromal cells (WPSCs), (iv) whole chorion derived stromal cells (CSCs), and (v) chorionic villi-derived stromal cells (CVCs), and a pharmaceutically acceptable carrier.

In another embodiment, the present invention provides PSC compositions as above-described which comprise at least two of the following cell types: (i) APSCs, (ii) PPSCs, (iii) WPSCs, (iv) CSCs, and (v) CVCs.

In another embodiment, the present invention provides PSC compositions as above-described which comprise at least three of the following cell types: (i) APSCs, (ii) PPSCs, (iii) WPSCs, (iv) CSCs, and (v) CVCs.

In another embodiment, the present invention provides PSC compositions as above-described which comprise at least four of the following cell types: (i) APSCs, (ii) PPSCs, (iii) WPSCs, (iv) CSCs, and (v) CVCs.

In another embodiment, the present invention provides PSC compositions as above-described which comprise all five of the following cell types: (i) APSCs, (ii) PPSCs, (iii) WPSCs, (iv) CSCs, and (v) CVCs.

In another embodiment, the present invention provides PSC compositions as above-described wherein at least one of the APSCs, PPSCs, WPSC, or CVCs express a molecular marker selected from the group consisting of CD105, CD90, CD73, CD273, CD210, and a combination thereof.

In another embodiment, the present invention provides PSC compositions as above-described wherein at least one of the APSCs, PPSCs, WPSCs, or CVCs does not express a molecular marker selected from the group consisting of CD11b, CD45, HLADR, CD119, CD85b, CD178, CD40, and a combination thereof.

In another embodiment, the present invention provides PSC compositions as above-described wherein said cells are derived from more than one donor.

In another embodiment, the present invention provides PSC compositions as above-described wherein said cells are derived from more than one donor of the same blood type.

In another embodiment, the present invention provides PSC compositions as above-described, wherein said cells are derived from more than one donor which have been determined to be histocompatible with each other.

In another embodiment, the present invention provides PSC compositions as above-described wherein said cells are derived from more than one donor which have been determined to comprise the same or comprise similar human leukocyte antigens (HLA) alleles or major histocompatibility complex (MHC).

In another embodiment, the present invention provides PSC compositions as above-described, wherein said cells are derived from one or more donors wherein the DNA thereof has been analyzed to confirm that the cells do not comprise gene mutations including those correlated to genetic diseases including but not limited to Autosomal dominant diseases such as familial hypercholesterolemia, Neurofibromatosis type I, Hereditary spherocytosis, Marfan syndrome, Huntington's disease, Autosomal recessive diseases such as Sickle cell anemia, Cystic fibrosis, Tay-Sachs disease, Phenylketonuria, Autosomal recessive polycystic kidney disease, Mucopolysaccharidoses, Lysosomal acid lipase deficiency, Glycogen storage diseases such as Galactosemia, X-linked diseases such as Duchenne muscular dystrophy, and Hemophilia.

In another embodiment, the present invention provides PSC compositions as above-described, wherein said cells are derived from one or more donors which have been determined not to comprise any pathogenic viruses or other microbial pathogens.

In another embodiment, the present invention provides a method of reducing chronic inflammation and consequent tissue fibrosis in a subject comprising administering to the subject in need thereof the perinatal stromal cell (PSC) composition of any one of the foregoing claims.

In another embodiment, the present invention provides a method of treating or preventing fibrosis in a subject in need thereof comprising administering to the subject in need thereof the perinatal stromal cell (PSC) composition of any one of the foregoing claims.

In another embodiment, the present invention provides methods as afore-described, wherein the subject comprises lung or pulmonary fibrosis, e.g., caused by infection, chemotherapy, cancer, COPD, environmental insults such as asbestos, coal dust and the like, or a disease such as cancer or cystic fibrosis; liver fibrosis e.g., caused by alcoholism, fatty liver disease, NASH, hepatitis B or hepatitis C; heart fibrosis e.g., caused by disease, infection, heart attack or stroke; Mediastinal fibrosis characterized by calcified fibrosis of the lymph nodes; retroperitoneal cavity fibrosis; bone marrow fibrosis; skin fibrosis; or scleroderma or systemic sclerosis.

In another embodiment, the present invention provides methods as afore-described, wherein the fibrotic tissue comprises a lung, liver, heart, pancreas, blood vessel, large intestine, small intestine, kidney, skin, interstitium, or a scar tissue.

In another embodiment, the present invention provides methods as afore-described, wherein a collagen content and or a collagen deposit in the tissue is reduced as compared to the collagen content or collagen deposit in said tissue before the administration of the PSC composition.

In another embodiment, the present invention provides methods as afore-described, wherein a fibrotic score in the tissue is decreased as compared to said fibrotic score in the tissue before the administration of the PSC composition.

In another embodiment, the present invention provides methods as afore-described, wherein the fibrotic score is selected from the group consisting of an Ashcroft score and an Ishak score.

In another embodiment, the present invention provides methods as afore-described, wherein a molecular marker of fibrosis is decreased in the tissue as compared to said molecular marker in the tissue before the administration of the PSC composition, optionally wherein the molecular marker of fibrosis is selected from the group consisting of αv-integrin expression, MMP-2 activity, pAKT/AKT expression ratio, miR199 expression, and a combination thereof and/or optionally wherein an anti-fibrotic molecular marker is increased in the tissue as compared to said molecular marker in the tissue before the administration of the PSC composition, further optionally wherein the anti-fibrotic molecular marker is Caveolin-1 expression.

In another embodiment, the present invention provides a method of reducing or preventing tissue or organ inflammation in a subject in need thereof comprising administering to the subject in need thereof any of the afore-described perinatal stromal cell (PSC) compositions.

In another embodiment, the present invention provides a method of reducing or preventing tissue or organ inflammation in a subject in need thereof comprising administering to the subject in need thereof any of the afore-described perinatal stromal cell (PSC) compositions, wherein a molecular marker of inflammation in a tissue is decreased as compared to said molecular marker in the tissue before the administration of the PSC composition, optionally wherein the molecular marker of inflammation is selected from the group consisting of TNFα expression, INFγ expression, IL-17 expression, and a combination thereof.

In another embodiment, the present invention provides a method of reducing or preventing tissue or organ inflammation in a subject in need thereof comprising administering to the subject in need thereof any of the afore-described perinatal stromal cell (PSC) compositions, wherein CD45+ T cell infiltration in a tissue is decreased as compared to before the administration of the PSC composition.

In another embodiment, the present invention provides a method of inducing immune tolerance in a subject comprising administering to the subject in need thereof any of the afore-described perinatal stromal cell (PSC) compositions, optionally wherein the proliferation of pro-inflammatory mature monocyte-derived dendritic cells (moDC) is inhibited, the proliferation of tolerogenic immature moDC is induced, the proliferation of pro-inflammatory CD4+, CD8+, CD3+, CD4+CD8+(double positive), and/or CD25+ T cells is inhibited, and/or the proliferation of CD11b+, CD11c+ T cells is inhibited and/or optionally wherein a decreased expression of maturity markers CD1a and CD83 in a monocyte population indicates an inhibition of mature moDC proliferation, and wherein an increased expression of immaturity markers CD85d and CD14 in a monocyte population indicates an increase of immature moDC proliferation.

In another embodiment, the present invention provides any of the afore-described methods wherein the subject has single or multi-organ fibrosis, idiopathic pulmonary fibrosis (IPF), interstitial lung disease (ILD), dilated cardiomyopathy (DCM), or graft versus host disease (GVHD).

In another embodiment the invention provides a method of treating single- or multi-organ fibrosis, idiopathic pulmonary fibrosis (IPF), interstitial lung disease (ILD), dilated cardiomyopathy (DCM), or graft versus host disease in a subject (GVHD) comprising administering to the subject in need thereof any of the afore-described perinatal stromal cell (PSC) compositions.

In another embodiment, the present invention provides any of the afore-described methods wherein the PSC composition comprises chorionic villi-derived stromal cells (CVC), and a pharmaceutically acceptable carrier.

In another embodiment, the present invention provides any of the afore-described methods wherein the subject has or is at risk of developing acute respiratory distress syndrome (ARDS) or sepsis.

In another embodiment, the present invention provides any of the afore-described methods wherein the subject has an acute or chronic viral disease or infection, e.g., hepatitis A, B, C, D or E or coronavirus infection such as SARS-CoV, SARS-CoV-2, MERS, and/or has an acute or chronic bacterial disease or infection, e.g., influenza or pneumococcal infection, optionally one that puts the subject at risk of developing acute respiratory distress syndrome (ARDS) or sepsis or fibrosis.

In another embodiment, the present invention provides any of the afore-described methods wherein the subject has tissue or organ inflammation, e.g., pericarditis.

In another embodiment, the present invention provides any of the afore-described methods wherein the subject has or is at risk of developing tissue or organ inflammation caused by a vaccine, optionally an mRNA vaccine.

### BRIEF DESCRIPTION OF THE DRAWINGS

FIG. 1A illustrates a Xenogeneic Graft versus Host Disease (GVHD) induction diagram. huPBMC: Human Peripheral Blood Mononucleated Cells, huPSC: Human Perinatal Stromal Cells. FIG. 1B shows histograms illustrating Perinatal Stromal Cell flow cytometry characterization. APSC: Amniotic Perinatal Stromal Cells, PPSC: Placental Proper Stromal Cells, WPSC: Wharton's Jelly Perinatal Stromal Cells. FIG. 1C shows tissue cultured isolated cells. Scale bar=400 μm.

FIG. 2A is a graph bar illustrating flow cytometry results of human specific CD3+ cells from mice samples at day 40. FIG. 2B is a graph illustrating daily percent weight change per experimental condition. FIG. 2C is a graph bar illustrating area under the curve of total body weight. FIG. 2D is a graph bar illustrating Total mean percentage body change. ns=P>0.05, *=P≤0.05, **=P≤0.01, ***=P≤0.001. PBMC and Cyclosporin A (CsA) are the respective positive (GvHD) and negative (attenuated GvHD) controls.

FIG. 3A is a graph illustrating the time to return to baseline (zero change) of weight change. FIG. 3B is a graph illustrating AUC of GvHD scores. *=P≤0.05, **=P≤0.01, ***=P≤0.001. PBMC and Cyclosporin A (CsA) are the respective positive (GvHD) and negative (attenuated GvHD) controls.

FIG. 4A shows histological samples of mice lung tissue stained for human specific hCD45+ cells for: GvHD (PBMC positive control), Cyclosporin A (CsA negative control), Placental Proper Stromal Cell (PPSC), Amniotic Perinatal Stromal Cell (APSC), Wharton's Perinatal Stromal Cells (WPSC). Upper row scale=1 mm and lower row scale=200 μm.

FIG. 4B is a graph illustrating the percentage of human CD45+ cell in lung tissue. N=3. *P<0.05. **P<0.001.

FIG. 5A shows histological samples of mice liver tissue stained for human specific CD45+ cells for: GvHD (PBMC positive control), Cyclosporin A (CsA negative control), Placental Perinatal Stromal Cell (PPSC), Amniotic Perinatal Stromal Cell (APSC), Wharton's Perinatal Stromal Cells (WPSC). Upper row scale=1 mm and lower row scale=200 μm. FIG. 5B is a graph illustrating the percentage of human CD45+ cell in liver tissue. N=3. *P<0.05. **P<0.001.

FIG. 6A shows histological samples of mice lung tissue stained with Masson's Trichrome. Upper row scale=1 mm and lower row scale=200 μm. FIG. 6B is a graph illustrating the fibrotic pathological scores (Ashcroft) for: GvHD (PBMC positive control), Cyclosporin A (CsA negative control), Placental Proper Stromal Cell (PPSC), Amniotic Perinatal Stromal Cell (APSC), Wharton's Perinatal Stromal Cells (WPSC). N=3. *P<0.05.

FIG. 7A shows histological samples of mice liver tissue stained with Masson's Trichrome. Upper row scale=1 mm and lower row scale=200 μm. FIG. 7B is a graph illustrating the fibrotic pathological scores (Ishak) for: GvHD (PBMC positive control), Cyclosporin A (CsA negative control), Placental Perinatal Stromal Cell (PPSC), Amniotic Perinatal Stromal Cell (APSC), Wharton's Perinatal Stromal Cells (WPSC). N=3. *P<0.05. **P<0.001.

FIG. 8A is a curve illustrating the percentage survival for the duration of the study (55 days) including animals suffering of severe weight loss (>30%). FIG. 8B is a curve illustrating the percentage survival for the duration of the study (55 days) including animals with weight loss <15%). GvHD (PBMC positive control), Cyclosporin A (CsA negative control), Placental Proper Stromal Cell (PPSC), Amniotic Perinatal Stromal Cell (APSC), Wharton's Perinatal Stromal Cells (WPSC). N=10. *P<0.05.

FIG. 9 is a curve illustrating the percentage survival of mice with bleomycin (BLM)-induced lung injury administered with saline, allogeneic adipose (ASCs), whole cord or Wharton's jelly (WJ) derived mesenchymal stem cells.

FIG. 10A is a histological sections of lung tissue of mice administered with saline (control), and stained with Masson's-Trichrome. FIG. 10B is a histological sections of lung tissue of BLM-treated mice stained with Masson's-Trichrome. FIG. 10C is a histological sections of lung tissue of mice infused with ASCs stained with Masson's-Trichrome. FIG. 10D is a histological sections of lung tissue of mice infused with CSCs stained with Masson's-Trichrome. FIG. 10E is a histological sections of lung tissue of mice infused with CVCs stained with Masson's-Trichrome. FIG. 10F is a histological sections of lung tissue of mice infused with WJ stained with Masson's-Trichrome. FIG. 10G is a graph illustrating the degree of pulmonary fibrosis on histological sections as measured by semi-quantitative Ashcroft score FIG. 10H is a graph illustrating the effect of the intratracheal BLM instillation on lung collagen content as measured by hydroxyproline assays Data are graphed as mean standard error of the mean (saline n=3, all other n=7-11/group). *P<0.05; **P<0.01.

FIG. 11A illustrates the ratio of phosphorylated AKT to AKT protein expression in lung tissue of subjects was quantified by western analysis at 21-day sacrifice. Data are graphed as mean±standard error of the mean (n=4-9/group). *P<0.05; **P<0.01. FIG. 11B illustrates MMP-2 activity as evaluated by zymography performed on protein extracts from lung tissue from BLM or BLM+ASC or BLM+CSC, BLM+CVC, BLM+WJ treated mice. Data are graphed as the mean±standard error of the mean of n=5-7/group *P<0.05. FIG. 11C illustrates Cav-1 protein expression as determined by western analysis. Data are graphed as mean±standard error of the mean (n=3-5/group). *P<0.05.

FIG. 12A is a graph bar illustrating the expression of the markers CD105, CD90, CD73, CD273, CD210, CD178, CD119, CD85d, CD40, CD11b, HLADR, and CD45 by villi stromal cells. FIG. 12B discloses histological sections of chorionic villus tissue illustrating the expression of the CD163, CD11b, PanCK, and PDL1 markers.

FIG. 13A is a graph bar illustrating the change in the number of mature moDC CD1a+ cells in the presence of villi stromal cells. FIG. 13B is a graph bar illustrating the change in the number of mature moDC CD83+ cells in the presence of villi stromal cells. FIG. 13C is a graph bar illustrating the change in the number of immature moDC CD85d+ cells in the presence of villi stromal cells. FIG. 13D is a graph bar illustrating the change in the number of immature moDC CD14+ cells in the presence of villi stromal cells.

FIG. 14A is a graph bar illustrating the change in the number of CD4+ cells after coculture of PBMC and villi stromal cells. FIG. 14B is a graph bar illustrating the change in the number of CD25+ cells after co-culture of PBMC and villi stromal cells. FIG. 14C is a graph bar illustrating the change in the number of CD8+ cells after co-culture of PBMC and villi stromal cells. FIG. 14D is a graph bar illustrating the change in the number of CD4+ and CD8+ cells after co-culture of PBMC and villi stromal cells. FIG. 14E is a graph bar illustrating the change in the number of CD3+ cells after co-culture of PBMC and villi stromal cells.

FIG. 15 is a graph bar illustrating the proliferation of T cells co-cultures with IL10-derived and villi-derived moDC cells.

FIG. 16A is a graph illustrating daily percent weight change per experimental condition. FIG. 16B is a graph illustrating AUC of GvHD scores. *=P≤0.05. PBMC and Cyclosporin A (CsA) are the respective positive (GvHD) and negative (attenuated GvHD) controls.

FIG. 17A shows histological samples of mice lung tissue stained with Masson's Trichrome. FIG. 17B is a graph illustrating the fibrotic pathological scores (Ashcroft) for: GvHD (PBMC positive control), and villi stromal cells *P<0.05. FIG. 17C shows histological samples of mice lung tissue stained for human specific hCD45+ cells for GvHD (PBMC positive control), and villi stromal cells. FIG. 17D is a graph illustrating the percentage of human CD45+ cell in lung tissue. *P<0.05. **P<0.001.

FIG. 18A shows histological samples of mice liver tissue stained for human specific hCD45+ cells for: GvHD (PBMC positive control), and villi stromal cells. FIG. 18B is a graph illustrating the percentage of human CD45+ cell in liver tissue. *P<0.05. **P<0.001. FIG. 18C shows histological samples of mice liver tissue stained with Masson's Trichrome. FIG. 18D is a graph illustrating the fibrotic pathological scores (Ishak) for GvHD (PBMC positive control), and villi stromal cells *P<0.05.

FIG. 19A illustrates histological samples of mice heart tissue sections stained with Masson's Trichrome. FIG. 19B is a graph illustrating the disease scores (Mean±SD)*, P value.

FIG. 20A illustrates the frequencies of IFN-γ producing T cells. FIG. 20B illustrates the frequencies of IL-17 producing T cells. (Mean±SD)*, P value.

FIG. 21 illustrates shows histological samples of mice lung tissue stained with Masson's Trichrome.

FIG. 22A illustrates Fibrosis score measured in mice after treatment. FIG. 22B illustrates collagen amount used to determine fibrosis index. FIG. 22C illustrates the mRNA level of expression of integrin after treatment. Bleomycin (BLM), Chorion Stromal Cells (Chorion), Villi Stromal Cells (Villi) and human Adipose Stromal Cells (hASC). Each point represents an individual mouse. *p<0.05, **p<0.001 compared to BLM treated mice.

### DETAILED DESCRIPTION OF THE INVENTION

The present invention is based on the seminal discovery that perinatal stromal tissues cells isolated from amnion, placenta, Wharton's jelly, chorionic membrane, chorionic villi, and umbilical cord tissue are useful for the treatment and/or prevention of graft versus host disease, and specifically for the treatment and/or prevention of tissue fibrosis such as idiopathic pulmonary fibrosis and multi-organ fibrosis.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a perinatal stromal cell (PSC) composition including amnion perinatal stromal cell (APSC), placenta proper stromal cell (PPSC), Wharton's jelly perinatal stromal cell (WPSC), whole chorion derived stromal cell (CSC), or chorionic villi-derived stromal cell (CVC), and a pharmaceutically acceptable carrier.

As used herein, “perinatal stromal cell,” or “PSC” refers to cells isolated from a placenta, preferably a human placenta. The human placenta includes a umbilical cord, a amnion membrane, and a “placenta proper”, which includes the chorion or chorionic plate, the villus, the intervillous space, the basal plate and the cotyledon. Each portion of the placenta can be isolated, and can be used to derive subpopulations of perinatal stromal cells.

The amnion membrane can be mechanically separated from the chorion, which leads to the derivation of amnion perinatal stromal cell (APSC). When sectioned longitudinally, the umbilical cord exposes Wharton's jelly, containing umbilical arteries and vein. After removal of the blood vessels, Wharton's Jelly perinatal stromal cell (WPSC, WJPSC, or MJ-MSC) can be derived from the umbilical cord. When the amnion and the umbilical cord are removed, the remaining portion of the placenta, which can be referred to as the placenta proper, can be used directly to prepare placenta proper stromal cell (PPSC), or can be further separated. For example, the chorionic membrane can be detached to isolate whole chorion derived stromal cell (CSC), and the intermediate and terminal villi can be exposed to isolate chorionic-villi stromal cell (CVC).

Wharton's jelly (WJ) is an ideal reservoir to obtain PSCs for use in clinical applications because these PSCs maintain more stem-like properties than SCs from other sources, such as adipose tissue. No ethical issues arise with the use of these non-embryonic MSCs. Since impaired pulmonary repair mechanisms associated with aging likely underlie the pathogenesis of IPF, this younger source of MSCs may confer certain advantages over ASCs in the treatment of BLM-induced IPF in aging mice. In addition, umbilical cord-derived cells are easy to harvest because the source is readily available and typically rendered as waste.

In experimental models, WJ-derived MSCs exhibit anti-fibrotic effects. In a unilateral ischemia-reperfusion injury rat model of renal fibrosis, a purported mechanism involves the delay of epithelial-to-mesenchymal transition and rescue of renal fibrosis. WJ-MSCs have also been studied as a treatment for S. mansoni-induced liver fibrosis. Upon transplantation of WJ-MSCs, it was reported that WJ-MSCs differentiated into hepatocyte-like cells expressing markers specific to human hepatocytes. Further, liver fibrosis regressed coinciding with down-regulation of collagen I, alpha smooth muscle actin, and IL-13, markers related to hepatic fibrosis. The profibrotic profile of human keloid fibroblasts was enhanced by co-culture with WJ-MSC conditioned media. In contrast WJ-MSC conditioned media promoted normal skin fibroblast migration and wound closure.

By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid, vitamin A, vitamin E, and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, cysteine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG); retinyl palmitate, selenium, methionine, citric acid, sodium sulfate and parabens. Examples of diluent include, but are not limited to, water, alcohol, saline solution, glycol, mineral oil and dimethyl sulfoxide (DMSO).

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation.

The pharmaceutical composition may further contain additional pharmaceutical or therapeutic agent, as evaluated beneficial by the physician administering said pharmaceutical composition.

In one aspect, the APSC, PPSC, WPSC, or CVC expresses a molecular marker selected from the group consisting of CD105, CD90, CD73, CD273, CD210, and a combination thereof. In another aspect, the APSC, PPSC, WPSC, or CVC does not express a molecular marker selected from the group consisting of CD11b, CD45, HLADR, CD119, CD85b, CD178, CD40, and a combination thereof.

As used herein “molecular marker” refers to a characteristic of feature of a tissue, or a cell, that can be assessed, and which provides useful information regarding the tissue or cell. Non-limiting example of molecular marker include, but are not limited to, the expression or activity of a gene, a protein, a miRNA, a mRNA, an enzyme, etc. The expression of a membrane-associated protein can be a molecular marker; and the expression or lack thereof of the protein can be useful for the identification of the cell type. The activity of an enzyme can be a molecular marker; and the activity of the enzyme, or lack thereof, can be useful to identify pathway activation or repression in a cell. The expression of a gene, a mRNA, or miRNA can be a molecular marker; and the expression or lack thereof of the gene, mRNA, or miRNA can be useful to identify pathway activation or repression in a cell.

In another embodiment, the invention provides a method of reducing chronic inflammation and consequent tissue fibrosis in a subject including administering to the subject in need thereof a perinatal stromal cell (PSC) composition of the invention.

Fibrosis is the formation of excess fibrous connective tissue in an organ or tissue in a reparative or reactive process. Physiologically, fibrosis acts to deposit connective tissue, which can interfere with or totally inhibit the normal architecture and function of the underlying organ or tissue. Fibrosis can be used to describe the pathological state of excess deposition of fibrous tissue, as well as the process of connective tissue deposition in healing. Fibrosis can be defined by the pathological accumulation of extracellular matrix (ECM) proteins, and results in scarring and thickening of the affected tissue, which interferes with normal organ function. Fibrosis can occur virtually in any tissue within the body, as a result of inflammation or damage to said tissue. Non-limiting examples of tissue that can be affected by fibrosis include: lung, liver, brain, kidney, artery, intestine, joint (knee, shoulder), skin, hand, finger, soft tissue, penis, and heart.

In one aspect, the tissue is a lung, liver, heart, pancreas, blood vessel, large intestine, small intestine, kidney, skin, interstitium, or a scar tissue.

As used here “reducing tissue fibrosis in a subject” can refer to any intervention that cures, slows down, lessens symptoms of, and/or halts progression of the fibrosis processes occurring in a tissue.

The term “subject” as used herein refers to any individual or patient to which the subject methods are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., reduction of tissue fibrosis, reduction of tissue inflammation, increase of immune modulation).

In some aspects, a collagen content and or a collagen deposit in the tissue is reduced as compared to the collagen content or collagen deposit in said tissue before the administration of the PSC composition.

Fibrosis is defined by the overgrowth, hardening, and/or scarring of various tissues and is attributed to excess deposition of extracellular matrix components including collagen. Therefore, evaluating the collagen content of a tissue, and/or any changes in the deposition of collagen in a tissue is a useful technique to evaluate and monitor fibrosis. Masson's trichrome staining, which is a three-color staining protocol used in histology allows distinguishing cells from surrounding connective tissue, by staining keratin and muscle fibers in red, collagen and bone in blue or green, cytoplasm in light red or pink, and cell nuclei in dark brown to black. Other methods that allows the detection and quantification of collagen in a tissue exist in the art, and any suitable method can be used to that effect.

In other aspects, a fibrotic score in the tissue is decreased as compared to said fibrotic score in the tissue before the administration of the PSC composition.

As used herein “fibrotic score” refers to any validated scoring method that can be used to easily and reproducibly evaluate the amount of fibrosis in a tissue. Depending on the tissue, and/or on the disease affecting the tissue, different scoring methods can be applied.

In many aspects, the fibrotic score is selected from the group consisting of an Ashcroft score and an Ishak score.

The Ashcroft score is a continuous numerical scale created for determining the degree of fibrosis in lung specimens and for correlation with other pulmonary variables such as lung function tests or mineral burden. Grading is scored on a scale from 0 to 8, using the average of microscope field scores. The system allows fibrosis to be measured in small samples of tissue (1 cm) which can provide a detailed description of the changes in a lung, currently not possible with most existing methods.

Several scoring system exist for liver fibrosis evaluation, including IASL, Batts-Ludwig, Metavir, and Ishak scores. Ishak is one of the most widely accepted scoring systems for assessment of fibrosis and necro-inflammation in dealing with chronic hepatitis C.

In one aspect, a molecular marker of fibrosis is decreased in the tissue as compared to said molecular marker in the tissue before the administration of the PSC composition.

Scoring systems allow for the global evaluation of a tissue, usually by studying histological tissue samples. Molecular marker of fibrosis can also be used to evaluate the levels of expression of protein, enzyme, or miRNA that are involved in molecular pathways capable of regulating fibrosis. Molecular markers can be pro-fibrotic, an increase in their expression/activity can thus indicate an increase in fibrosis; or molecular markers can be anti-fibrotic, an increase in their expression/activity can thus indicate a decrease in fibrosis. For examples αᵥ-integrin expression, MMP-2 activity, pAKT/AKT ratio, and miR199 expression are known in the art to be markers of fibrosis. Caveolin-1 is known in the art to be an anti-fibrotic marker.

In various aspects, the molecular marker of fibrosis is selected from the group consisting of αᵥ-integrin expression, MMP-2 activity, pAKT/AKT expression ratio, miR199 expression, and a combination thereof. In another aspect, an anti-fibrotic molecular marker is increased in the tissue as compared to said molecular marker in said tissue before the administration of the PSC composition. In various aspects, the anti-fibrotic molecular marker is Caveolin-1 expression.

In an additional embodiment, the invention provides a method of reducing tissue inflammation in a subject including administering to the subject in need thereof a perinatal stromal cell (PSC) composition of the invention.

An “inflammatory response” or “inflammation” occurs when tissues are injured by bacteria, trauma, toxins, heat, or any other cause. The damaged cells release chemicals including histamine, bradykinin, and prostaglandins causing swelling. The chemicals also attract white blood cells such as phagocytes to eliminate germs and dead or damaged cells. Tissue damage and inflammation are important triggers for regeneration and fibrosis by inducing the recruiting and activation a variety of different cells types of the innate and adaptive immune system.

In one aspect, a molecular marker of inflammation in the tissue is decreased as compared to said molecular marker in the tissue before the administration of the PSC composition.

In some aspects, the molecular marker of inflammation is selected from the group consisting of TNFα expression, INFγ expression, IL-17 expression, and a combination thereof.

The tumor necrosis factor (TNF) superfamily refers to a superfamily of cytokines that can cause cell death. All TNF superfamily members form homotrimeric (or heterotrimeric in the case of LT-alpha/beta) complexes that are recognized by their specific receptors. Examples of TNF super family members include TNFα, TNF-P, lymphotoxin-alpha, CD40L, CD27L, CD30L, FASL, 4-1BBL, OX40L and TRAIL.

Interferons (IFNs) are a group of signaling proteins made and released by host cells in response to the presence of several pathogens, such as viruses, bacteria, parasites, and also tumor cells. In a typical scenario, a virus-infected cell will release interferons causing nearby cells to heighten their anti-viral defenses. IFNs belong to the large class of proteins known as cytokines, molecules used for communication between cells to trigger the protective defenses of the immune system that help eradicate pathogens. Examples of IFNs include IFN-α, IFN-β, IFN-ε, IFN-κ and IFN-γ.

Interleukin 17A (IL-17 or IL-17A) is a pro-inflammatory cytokine produced by a group of T helper cell known as T helper 17 cell in response to their stimulation with IL-23.

Infiltration of T lymphocytes in tissue is common in patients with and in animal models of fibrosis. The cells are believed to play a role of the in regulating the accumulation of extracellular matrix, particularly collagen. Both profibrotic and antifibrotic T lymphocytes can be identified as playing a role in fibrosis. Elimination of profibrotic infiltrating T lymphocytes can be approached to improving outcomes in patients with fibrosis.

In other aspects, CD45+ T cell infiltration in the tissue is decreased as compared to before the administration of the PSC composition.

In a further embodiment, the invention provides a method of inducing immune tolerance in a subject including administering to the subject in need thereof a perinatal stromal cell (PSC) composition of the invention.

An “immune response” refers to an integrated bodily response to an antigen and preferably refers to a cellular immune response or a cellular as well as a humoral immune response. The immune response may be protective/preventive/prophylactic and/or therapeutic or pathologic. The immune system is a system of biological structures and processes within an organism that protects against disease. This system is a diffuse, complex network of interacting cells, cell products, and cell-forming tissues that protects the body from pathogens and other foreign substances, destroys infected and malignant cells, and removes cellular debris: the system includes the thymus, spleen, lymph nodes and lymph tissue, stem cells, white blood cells, antibodies, and lymphokines. B cells or B lymphocytes are a type of lymphocyte in the humoral immunity of the adaptive immune system and are important for immune surveillance. T cells or T lymphocytes are a type of lymphocyte that plays a central role in cell-mediated immunity. There are two major subtypes of T cells: the killer T cell and the helper T cell. In addition, there are suppressor T cells which have a role in modulating immune response. Killer cells only recognize antigens coupled to Class I MHC molecules, while helper T cells only recognize antigens coupled to Class II MHC molecules. These two mechanisms of antigen presentation reflect the different roles of the two types of T cell. A third minor subtype are the γδ T cells that recognize intact antigens that are not bound to MHC receptors. In contrast, the B cell antigen-specific receptor is an antibody molecule on the B cell surface, and recognizes whole pathogens without any need for antigen processing. Each lineage of B cell expresses a different antibody, so the complete set of B cell antigen receptors represent all the antibodies that the body can manufacture.

A “cellular immune response”, a “cellular response”, a “cellular response against an antigen” or a similar term is meant to include a cellular response directed to cells characterized by presentation of an antigen with class I or class II MHC. The cellular response relates to cells called T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells. In preferred embodiments, the present invention involves the stimulation of an anti-tumor CTL response against tumor cells expressing one or more tumor expressed antigens and preferably presenting such tumor expressed antigens with class I MHC.

The terms “immunoreactive cell” “immune cells” or “immune effector cells” in the context of the present invention relate to a cell which exerts effector functions during an immune reaction. An “immunoreactive cell” preferably is capable of binding an antigen or a cell characterized by presentation of an antigen or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

“Immune regulation” or “immune tolerance” is fundamental to ensure that an immune response is appropriate. As used herein, “inducing immune tolerance” refers to the induction of regulatory cells that are responsible for maintaining a balanced and appropriate immune response. Immune-regulatory cells include regulatory T cells, B cells and macrophages, as well as myeloid-derived suppressor cells, dendritic cells and mesenchymal stromal cells (MSCs). These cells can modulate immune responses by inhibiting effector cells and by inducing other regulatory cells.

In one aspect, the proliferation of pro-inflammatory mature monocyte-derived dendritic cells (moDC) is inhibited, the proliferation of tolerogenic immature moDC is induced, the proliferation of CD4+, CD8+, CD3+, CD4+CD8+(double positive), and/or CD25+ T cells is inhibited, and/or the proliferation of CD11b+, CD11c+ T cells is inhibited.

Monocytes function as macrophage precursors, and have the capacity to differentiate into dendritic cells (DCs), therefore playing an essential role in both the innate and adaptive immunity. Mature monocyte-derived dendritic cells (moDC) express maturity markers such as CD1a and CD83, and have pro-inflammatory capability. Immature moDC, or tolerogenic DC express immaturity markers such as CD85d and CD14, and have immuno-suppressive properties.

In some aspects, a decreased expression of maturity markers CD1a and CD83 in a monocyte population indicates an inhibition of mature moDC proliferation; in other aspects, an increased expression of immaturity markers CD85d and CD14 in a monocyte population indicates an increase of immature moDC proliferation.

In many aspects, the subject has single- or multi-organ fibrosis, idiopathic pulmonary fibrosis (IPF), interstitial lung disease (ILD), dilated cardiomyopathy (DCM), or graft versus host disease (GVHD).

In some aspects, the subject has scleroderma. As used herein, “scleroderma” refers to a group of autoimmune diseases that may result in changes to the skin, blood vessels, muscles, and internal organs. Specifically due to the increased synthesis of collagen (leading to the sclerosis), the damage to small blood vessels, the activation of T lymphocytes and the production of altered connective tissues, scleroderma lead to thickening, stiffness, and fibrosis is the skin, blood vessels, muscles, and internal organs.

As used herein, the term “single-organ fibrosis” is meant to refer to the treatment of individual organs separately affected by fibrosis (i.e., lung fibrosis, liver fibrosis, heart fibrosis, kidney fibrosis, etc.), that can each individually be treated using the compositions described herein; while the term “multi-organ fibrosis” refers to the treatment of several organs affected by fibrosis in a same subject at the same time. Indeed, in some instances, fibrosis can extend to multiples organs and not being confined to only one.

Idiopathic pulmonary fibrosis (IPF) is a chronic, progressive-fibrosing interstitial lung disease (ILD) of unknown origin characterized by progressive lung scarring and the histologic picture of usual interstitial pneumonia. Disorders belonging to the ILD category cause damage to the lung interstitium through various mechanisms, including inflammation, edema, and/or fibrosis. According to most recent estimates, there are over 200,000 Americans suffering from pulmonary fibrosis. IPF presents mostly in men over the age of 60. Faced with an unpredictable, relentless clinical course, patients are offered management involving the use of untargeted therapies that have limited efficacy and substantial morbidity. Historically, features of animal models of bleomycin (BLM)-induced pulmonary fibrosis have been used to study fibrotic lung disease that have led to direct translation of results to human IPF, including a Phase I safety trial for patient with IPF (ClinicalTrials.gov, NCT02013700). Recognizing the clinical burden of irreversible and disabling fibrotic lung disease drives the development of more efficacious therapies.

Dilated cardiomyopathy (DCM) is the leading cause of heart failure (HF) and heart transplantation in young adults; yet, despite how frequent and devastating this disease is, there is still no effective treatment to prevents or reverse this condition. DCM is a significant health care concern and accounts for approximately 10% of the 5.8 million cases of heart failure (HF) in the United States and over 23 million cases worldwide. The etiology of DCM is heterogeneous and includes genetic, non-genetic, and inflammatory causes. Inflammation is observed in at least 30-40% of DCM cases (DCMi) and therefore myocarditis is believed to be an important contributor to its pathogenesis. Myocarditis is more frequently in men and it is an important cause of sudden death in up to 20% of young adults. Because of its insidious onset it is widely underdiagnosed. The Myocarditis Treatment Trial showed a 1-year mortality rate of 20% and a 4-year mortality rate of over 50% in symptomatic patients; median survival of giant cell myocarditis is approximately 5 months upon disease onset. Current treatment of myocarditis and DCMi is primarily symptomatic and aimed at treating clinical manifestations of heart failure with angiotensin-converting enzyme (ACE) inhibitors, nitroglycerin, diuretics, and inotropic drugs in cases of severe HF. Immunosuppressive therapy is not effective in preventing or reversing DCMi even in cases where the underlying pathogenesis is inflammatory and autoimmune. The underlying reasons are poorly understood. An autoimmune mechanism for chronic myocarditis and progression to DCMi is supported by autoantibodies against heart tissue in myocarditis patients and the induction of experimental autoimmune myocarditis (EAM) with cardiac autoantigens in animal models. Progression of myocarditis to DCMi is a devastating complication of this disease and frequently results in fatal outcome. Inflammation is thought to play a critical role in progression of myocarditis to DCMi. For unknown reasons, treatment with immunosuppressive glucocorticoid (GC) drugs is not effective and does prevent progression of DCMi. Therefore, novel treatments to prevent or reverse DCMi are urgently needed.

Graft-versus-host disease (GvHD) is a chronic disease commonly associated with stem cell transplants such as those that occur with bone marrow transplants, but also applies to other forms of transplanted tissues such as solid organ transplants. White blood cells of the donor's immune system which remain within the donated tissue (the graft) recognize the recipient (the host) as foreign (non-self). The white blood cells present within the transplanted tissue then react against the recipient's body's cells, which leads to GvHD. This should not be confused with a transplant rejection, which occurs when the immune system of the transplant recipient rejects the transplanted tissue; GvHD occurs when the donor's immune system's white blood cells reject the recipient.

In yet another embodiment, the invention provides a method of treating single- or multi-organ fibrosis, idiopathic pulmonary fibrosis (IPF), interstitial lung disease (ILD), dilated cardiomyopathy (DCM), or graft versus host disease in a subject (GVHD) including administering to the subject in need thereof a perinatal stromal cell (PSC) composition of the invention.

The term “treating” is used to refer to a “treatment” or “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

In many aspects, the PSC composition includes chorionic villi-derived stromal cell (CVC), and a pharmaceutically acceptable carrier.

Presented below are examples discussing the perinatal stromal cells compositions contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Isolation and Characterization of Perinatal Stromal Cells

Perinatal stromal cells (PSCs) were isolated from human placenta, from which amnion membrane derived, umbilical cord derived, and chorion derived cells were isolated from healthy, term placentas collected by selective C-section after maternal consent and according to the guidelines of the ethical committee of the Cooperative Human Tissue Network at the University of Alabama. Human placental tissues were processed within 24 hours of collection in a sterile laminar hood as follows.

The amnion membrane was mechanically separated from the chorion and washed extensively with phosphate-buffered saline (PBS). It was then minced into small pieces and digested with Tryple (Gibco, Waltham, MA, USA) at 5 mL/g of tissue for 30 min in a shaker incubator (I24 Incubator Shaker series, New Brunswick Scientific, Edison, NJ, USA) at 37° C., 150 rpm to remove the amniotic epithelial cells. The undigested amnion was then removed, washed with PBS and further digested with 125 U/mg Collagenase I (Worthington, Lakewood, NJ, USA) at 37° C., 150 rpm for 1.5 h to isolate the amniotic mesenchymal cells (APSC). The mobilized cells in the digest were passed through a 100 μm cell strainer (VWR, Radnor, PA, USA) and collected by centrifugation at 500×g for 8 min.

The Wharton's Jelly (WPSC) was extracted from the umbilical cord as follows: the umbilical cord was sectioned in approximately 1.5 cm in length pieces and then dissected longitudinally to expose the Wharton's Jelly. The arteries and vein were removed, the remaining tissue was minced into small pieces, and digested with 125 U/mg Collagenase I at 37° C., 150 rpm for 2.5 h or until all tissue was digested. The digest was passed through a 100 μm cell strainer and centrifuged at 500×g for 8 min.

The placental proper was thoroughly washed with PBS, minced into small pieces, and digested with 125 U/mg Collagenase I at 37° C., 150 rpm for 1.5 h to isolate the placental proper stromal cells (PPSCs). The digest was passed through a 100 μm cell strainer and centrifuged at 500×g for 10 min to collect the PPSCs.

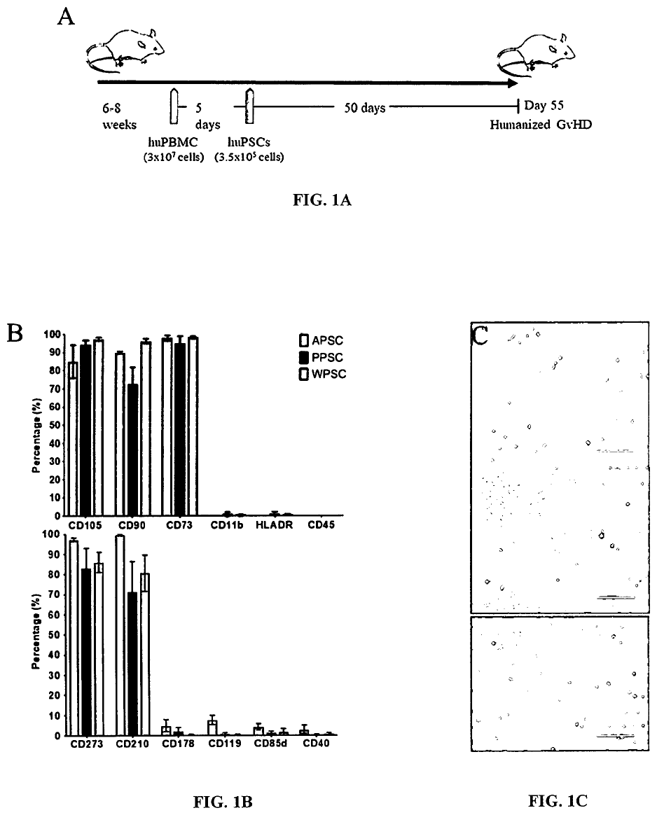

Freshly isolated perinatal stromal cells (PSCs) were cultured under standard tissue culture conditions (humidified, 37° C., and 5% CO₂) in MEM-alpha (Gibco, Waltham, MA, USA) supplemented with 1% Anti-Anti (Gibco) and 5% heat inactivated FBS (Gibco). The cell culture medium was replaced every other day and cells were sub-cultured when they reached 70-80% confluency. As illustrated in FIG. 1C, isolated cells from all perinatal stromal cells presented spindle like morphology when cultured.

PSCs were characterized by flow cytometry, to evaluate the expression of mesenchymal stromal cell related markers (CD105, CD90, CD73, CD11b, HLADR, and CD45) and other immune-relate markers (CD273, CD210, CD178, CD119, CD85d, and CD40).

PSCs were washed with Running buffer (Miltenyl Biotec Inc., Auburn, CA, USA) and centrifuged at 350×g for 5 minutes (Eppendorf, Westbury, NY, USA). The cells were incubated in blocking solution (Blockaid, Thermo, Austin, TX, USA) at 4° C. for 15 min. PSC samples (1×10⁵ cells/100 ul) were incubated with the following antibodies: CD85d-ILT4-PE, HLA-DR- TU36-PE, CD45-HI30-Brilliant Violet, CD73-AD2-PE (StemCell Technologies, Vancouver, BC, Canada), CD90- 5E10-PE (Molecular Probes, Eugene, OR), CD105-43A3-PE, CD273-B7DC-PE, CD119-IFNgRa-PE, CD40-5C3-FITC, CD11b-M1/70-FITC, and CD178-NOK-1-PE (Biolegend, San Diego, CA, USA), HLAG9-MEM-G/11-FITC (Invitrogen, Austin, TX, USA). Upon completion of the incubation, cells were washed twice with running buffer, centrifuged at 350×g, and then resuspended in 100 ul running buffer containing 300 μM DAPI (Biolegend) and incubated at 4° C. for 15 min in the dark. Whole blood samples were processed by centrifugation and red blood cell (RBC) were lysed with ACK buffer according to the manufacturer's instructions. The final single cell suspensions were prepared in Staining Buffer (PBS pH 7.4, 2.5% FBS, 0.09% NaN3) at 2×10⁷ cells/mL were added into 96-well plates and stained for 30 minutes at 4° C. with 100 μL of the reconstituted Live/Dead Aqua (Life Technologies) following manufacturer's instructions. After two washes with 150 μL of Staining Buffer, Fc receptors were blocked using TruStain Fc (Biolegend) in 100 μL volume for five to ten minutes on ice prior to immunostaining. Cells were stained for 30 min at 4° C. with antibodies, then washed twice with 150 μL of Staining Buffer and resuspended in 100 μL of Staining Buffer for analysis. Isotype control antibodies were used as negative staining controls when deemed necessary. All data were collected on a FortessaLSR (BD) and analyzed with FlowJo software (Tree Star, Inc.). Cell populations were defined according to the protocol and the gating strategy was determined by initial gating on singlets (FSC-H vs. FSCA), and then live cells based on Live/Dead Aqua viability staining. The percentage of human CD3+ (CD3 PE HIT3a, Biolegend) cells was determined according to the parent cell gate. Analysis were performed using Cytoflex flow cytometer (Beckman Coulter, Irving, TX, USA). As illustrated in FIG. 1B, PSCs have surface markers typically found in mesenchymal stromal cells (MSC) in addition to other suggested immune related markers used to identify such cells.

Perinatal Stromal Cell (PSCs) isolated herein had similar characteristics of Mesenchymal Stromal Cells (MSCs) based on the ISCT criteria, which is being plastic adherent under standard culture conditions, expression of CD105+, CD73+, CD90+, CD11b−, and CD45− HLADR. However, the capability of PSCs to differentiate into tri-lineage (Chondrocyte, Osteocyte and Adipocyte) was not evaluated, since it is not suspected that the effect of PSCs is based on their differentiation capabilities (stemness). Hence, PSCs were not referred to as MSCs. In addition, some other immune-pertinent markers were tested to further identify such cells. All PSCs were positive (>70%) for CD273+(PD-L2), CD210+(IL-10 Receptor) and negative (<5%) for CD178− (FasL), CD119− (IFNg Receptor), CD85d− (ILT4) and CD40. These additional immune-regulatory markers could be used to extend the characterization panel to identify such cells.

### Example 2

### Evaluation of the Effect of Perinatal Stromal Cells Infusion on Graft Versus Host Disease

The effect of perinatal stromal cells infusion on graft versus host disease were evaluated in a xenogeneic mice model of GVHD.

Non-obese diabetic/severe combined immunodeficient-IL-2 receptor gamma-null (NOD.Cg-Prkdcscid Il2rgtm1Wj1/SzJ, NSG®) mice were purchased from Jackson Laboratories (Bar Harbor, ME, USA). The mice were contained in a Charles River animal care facility, with food and water made available under pathogen-free conditions. The mice were exposed to a 12:12 hour light: dark cycle at controlled room temperature and humidity. All animal experiments were conducted with the approval of Charles River's Institutional Animal Care and Use Committee.

Xenogeneic GVHD NSG was generated in female NSG mice, which were engrafted intravenously (IV) via tail vein injection with 3×10⁷ human PBMCs and sorted into five groups of ten animals each. A positive control (PBMC) group received no treatment which allowed for GvHD development. A negative control (CsA) group was administered with Cyclosporin A once daily intraperitoneally (IP) at 15 mg/kg to study end (qd to end) from Day 1 to repress GvHD symptoms. On Day 5, three groups of treated mice received PSC infusions as a single dose of 350,000 of cells, delivered by intravenous tail vein injection (FIG. 1A). Cells were recovered from cryopreservation and allowed to recover in an incubator (humidified, 37° C., and 5% CO2) for 48 hours before injection. All cells used were between P2 and P3. Treatment groups were designated as follows: Amnion Perinatal Stromal Cells (APSC), Wharton's Jelly Perinatal Stromal Cells (WPSC), and Placental Proper Stromal Cells (PPSC).

Animal survival was monitored daily, weight and GvHD scoring was assessed three times a week. Clinical GVHD index was scored based on 5 parameters: weight loss, activity, posture, fur texture, and skin integrity; on a scale of 0 to 2, with 2 being most severe (Table 1). Animals were euthanized when their weight reached a weight loss of >30% or two consecutive loss measurements of >25%. Five animals in each group were analyzed at day 40 for expression of human CD3+ in whole blood (0.1 mL was collected by mandibular bleeds). Experiment was carried out until day 55.

TABLE 1
Clinical GVHD index scoring parameters

| Criteria | Grade 0 | Grade 1 | Grade 2 |
|---|---|---|---|
| Weight Loss | <10% | ≥10% < 25% | ≥25% |
| Activity | Normal | Mild to moderately decreased | Stationary until stimulated |
| Posture | Normal | Hunching only a rest | Severe gait or impared movement |
| Fur Texture | Normal | Mild to moderate ruffling | Severe rufflinlg/poor grooming |
| Skin Integrity | Normal | Scaling of paws and/or tail | Obvious areas of denuded skin |

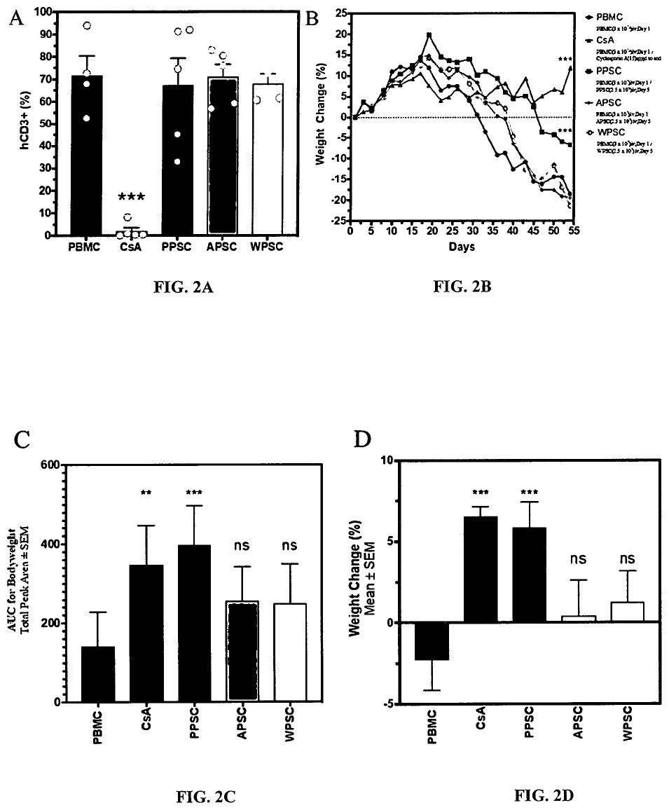

As illustrated in FIG. 2A, human PBMC were successfully engrafted in NSG mice, which was confirmed by the detection by flow cytometry of human specific CD3+ cells from mice blood samples at day 40. The negative control (Cyclosporin A) condition was the only sample with a significant reduction of hCD3+ compared to positive control (PBMC) and experimental conditions; no difference was observed between PBMC and experimental conditions. As shown in FIG. 2B GvHD was developed in engrafted mice, which was detected by weight loss, characteristic with the progression of the disease. Compared to PBMC, only the negative control and PPSC showed a significant difference in weight change by both percentage weight change, and area under the curve (AUC) of total body weight (FIGS. 2C-D).

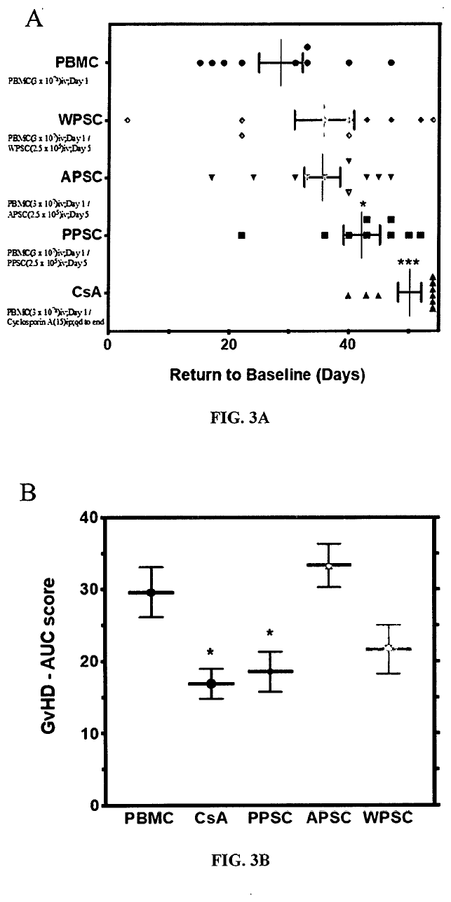

The return to baseline (days) was annotated for each individual animal per group as the day when their weight returned back to zero percent change (Day 0 weight) and graphed in order to calculate the rate of GvHD progression. In addition to this return to baseline number (FIG. 3A), the progression of GvHD was recorded using the scoring system described in Table 1, and the AUC per animal was graphed (FIG. 3B). The progression and rate of progression of GvHD was only significantly delayed in the Cyclosporin A and PPSC group.

Development of GVHD was also assessed using established histopathology, Masson's Trichrome stained slides, which quantitatively scored lung (Ashcroft) and liver (Ishak) fibrosis. Liver and lung (inflated) samples were collected from animals of each group as they reached endpoint. All organs were preserved in formalin for 24 h, transferred to 70% ethanol, and shipped at room temperature to Histowiz (Brooklyn, NY, USA) for processing to formalin-fixed paraffin-embedded (FFPE) blocks, H&E-stained slides, and special stains (Masson's Trichrome and human CD45+ staining). Certified pathologists (blinded) contracted by Histowiz provided histopathology scores for liver (Ishak) and lung (Ashcroft) fibrosis, and digitally quantified human CD45+ stained cells.

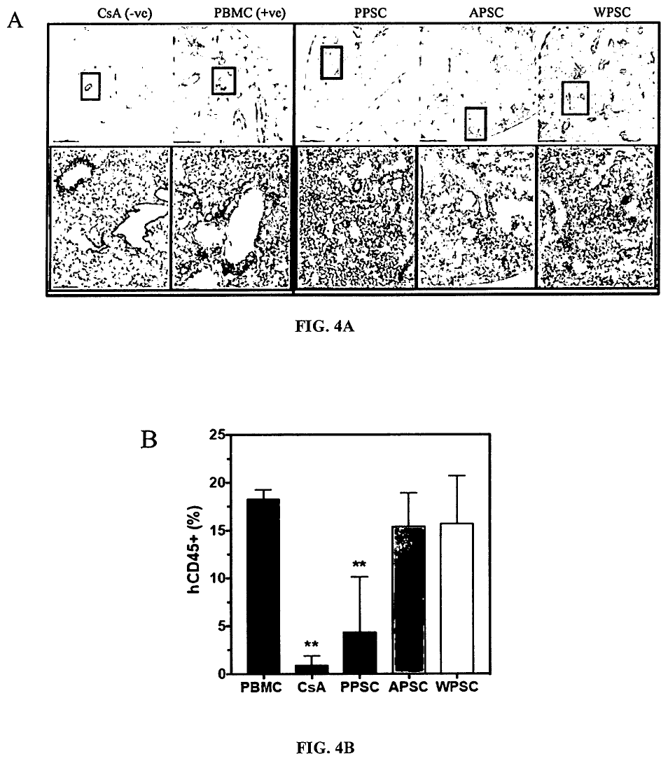

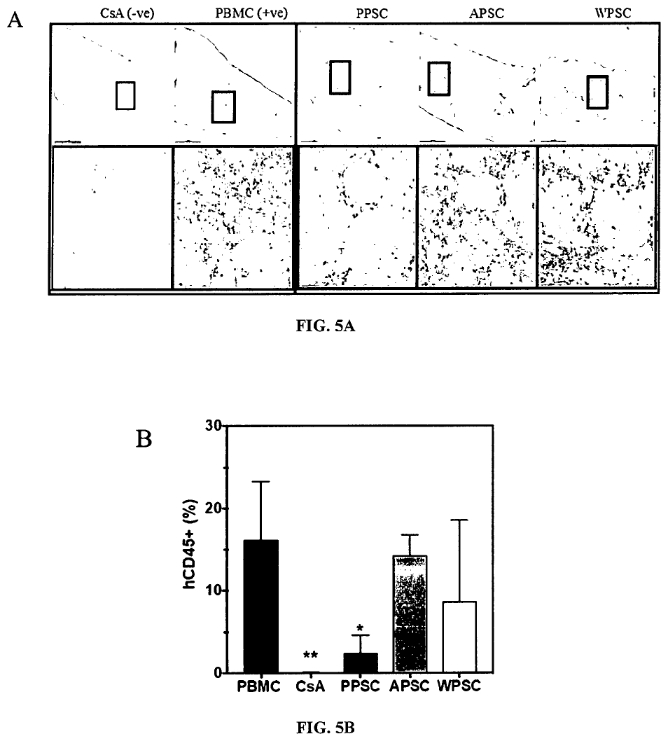

Human CD45+ cell infiltration was detected in histological samples for lung (FIG. 4) and for liver (FIG. 5) tissues. Quantification of human CD45+ stained cells (percentage) were calculated using image analysis. The majority of human CD45+ cells were located in surrounding the periphery of vascular lumen, which is a pattern typically seen with infiltrating cells from the vascular system. Compared to PBMC, only PPSC and negative control (CsA) showed a significant reduction of infiltrating human CD45+ cells into the tissue, which supports the above observations (GvHD score and weight loss).

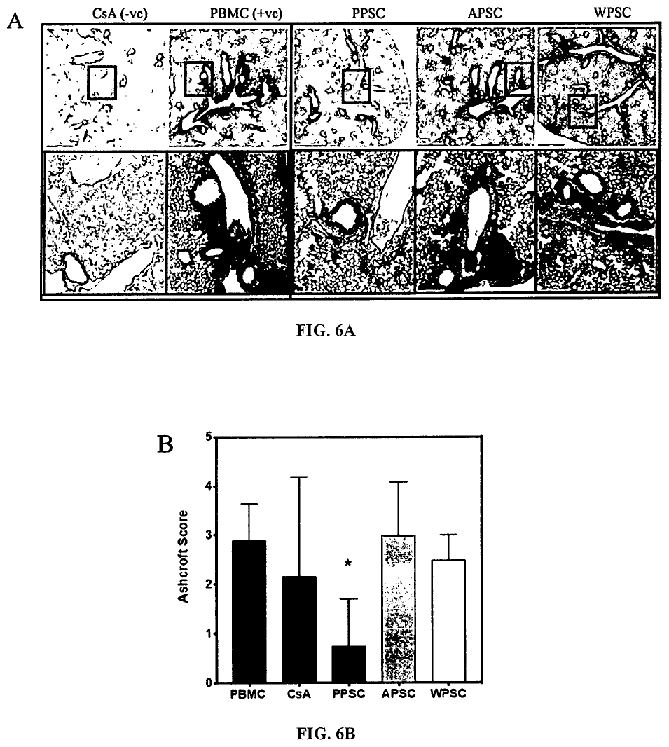

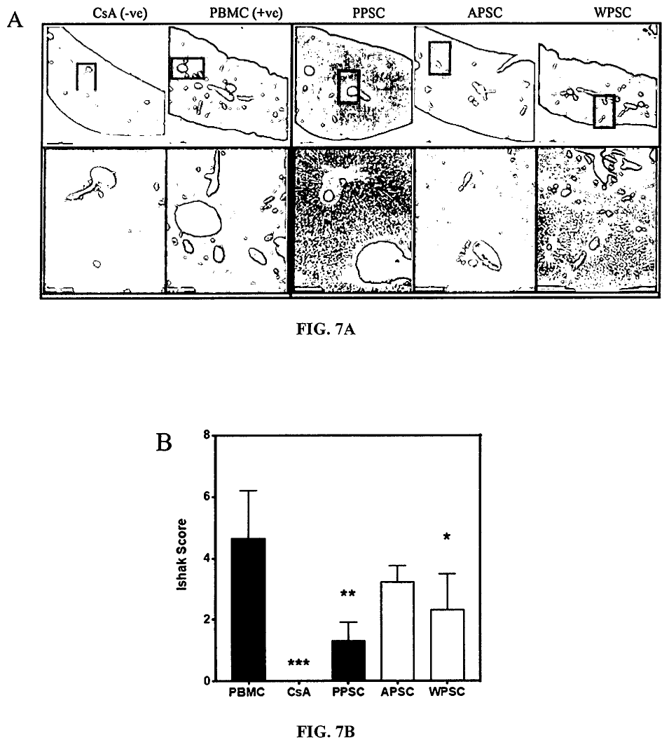

Histological sections of mice lung (FIG. 6) and liver (FIG. 7) stained with Masson's Trichrome (collagen depositions stained blue) determined that PPSC was the only treatment group that significantly reduced the fibrosis score for both lung and liver tissues compared to PBMC group, supporting the pathological fibrosis scores, Ashcroft and Ishak, for lung and liver respectively.

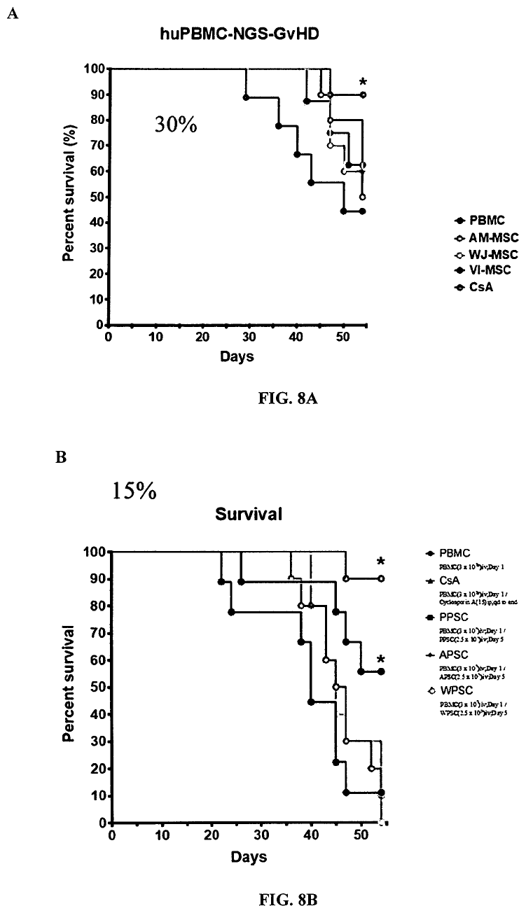

Survival analysis performed for 55 days showed that only the negative control (Cyclosporin A) and PPSC were significantly different compared to PBMC (FIG. 8). Most of the animals were sacrificed due to severe weight loss (>30%). Within the different treatment groups WPSC was he group that showed highest weight variability within the group (data not shown).

Evaluating the effect of cell therapeutics in a GvHD using a humanized model provided traditional objective measurements (body weight loss, GVHD scoring, fibrosis scoring etc.). In addition, it provided the opportunity to easily quantify human infiltrating cells that can be easily detected in mice tissue, which could provide better mechanistic chronic rejection studies in the future. Treatment with PPSC showed a significant reduction of infiltrating human CD45+ cells into the lung and liver; however, the percentage of circulating human CD3+ (FIG. 2A) was not different from the other treatment groups, suggesting that there is a possibility that PPSC are capable of changing the migratory or chemotactic activity of human CD45+ cells. Alternatively, it is also possible that PPSC could have an effect on endothelial cells and their capability to hinder activated human CD45+ cell migration.

Although the survival of the PPSCs was significantly different compared to PBMCs and the other experimental groups, it did not provide a permanent solution to GVHD since the weigh declined at day 45 compared to Cyclosporin A. This could be due to the limited effect of a single injection and perhaps the therapeutic effect could be maintained if multiple injections were administered during the course of the disease. Interestingly, the Cyclosporin A group had one animal that did not respond to the drug and succumbed to GVHD.

All treatments were well tolerated, but not all PSCs had the same therapeutic effect. PPSC cellular therapy demonstrated potential for efficacy toward GVHD based on various outcome measurements like GVHD score, weight loss, organ fibrosis and human CD45+ infiltration.

### Example 3

### Isolation and Characterization of PSCs

Wharton's Jelly (WJ) MSCs were derived from mothers that delivered male babies and tested positive for the Y chromosome as expected. Male derived adipose derived-MSCs (ASCs) from a 33-year-old male were purchased from Lonza (Lonza Bioscience, Walkersville MD). Cells were grown according to manufacturer's directions in Lonza media were used as a control.

WJ cells was isolated from the umbilical cord then cut into small pieces. The pieces were placed into several T-175 flask with minimum medium to allow attachment. The flasks were placed into a 37° C. incubator with 5% CO₂. The medium was composed of 10% fetal bovine serum (FBS) and 1% penicillin/streptomycin in α-MEM. Three days after initial plating, small amount of medium was added into the flasks to ensure the WJ pieces were attached securely. The cells were subsequently replenished with fresh medium every three to four days until the flasks reached 90% confluency. At confluency the cells were rinsed once with phosphate buffered saline (PBS) then incubated with trypsin for seven minute at 37° C. The trypsin was neutralized with medium and the cell layer centrifuged at 200×g for 10 minutes. The cells were counted and 1×10⁶ cells were seeded into each T-175 flask. The cells in the new flasks were designated as passage 1. The process was repeated to reach passage 3. When passage 3 cells reached confluency, the cells were harvested and cryopreserved at 1.25×10⁶ cells/mL of cryopreservation medium. The cryopreservation medium was composed of 10% FBS with 5% dimethysulfoxide (DMSO) in Hespan.

Chorionic membrane (CSC) and chorionic-villi (CVC) derived MSCs were isolated from healthy, term placentas collected by selective C-section after maternal consent and according to the guidelines of the ethical committee of the Cooperative Human Tissue Network at the University of Alabama (IRB protocol #940831016). Human placental tissues were processed within 24 hours of collection in a sterile laminar hood as follows. The chorion was mechanically separated from the amnion membrane and washed extensively with phosphate-buffered saline (PBS). The chorion membrane was then minced into small pieces and digested with 125 U/mg Collagenase I (Worthington, Lakewood, NJ, USA) at 37° C., 150 rpm for 1.5 h in a shaker incubator (124 Incubator Shaker series, New Brunswick Scientific, Edison, NJ, USA) to isolate the chorion stromal cells (CSCs). The mobilized cells in the digest were passed through a 100 μm cell strainer (VWR, Radnor, PA, USA) and collected by centrifugation at 500×g for 8 min. Finally, the placental proper tissue was carefully removed to expose the intermediate and terminal villi, dissected at the base of the intermediate villi and thoroughly washed with PBS, minced into small pieces, and digested with 125 U/mg Collagenase I at 37° C., 150 rpm for 1.5 h to isolate the chorionic villi stromal cells (VSCs). The digest was passed through a 100 μm cell strainer and centrifuged at 500×g for 8 min to collect the mobilized perinatal stromal cells (PSCs).

To confirm the fetal origin of the WJ derived-MSC isolated after the birth of male newborns, X/Y chromosome analysis was performed. Cells were analyzed at passage 2. Whole cord MSCs were assayed by PCR-mediated amplification and subsequent size analysis of short tandem repeats (STRs) to determine the maternal and/or fetal cell composition of cells or tissues. Genomic DNA was extracted from the cultured cells or tissue. This DNA was used in a multiplexed PCR-mediated amplification reaction targeting a total of fifteen autosomal STR and Amelogenin on the pseudo-autosomal region of the X and Y chromosomes. Following PCR amplification, fluorescently labeled PCR products were resolved by capillary electrophoresis on the ABI Genetic Analyzer. GeneMapper software (ABI) was used to calculate the number of repeats and relative abundance of each repeat for each STR locus. This data was then used to determine the absence or presence of maternal cells in the sample using the maternal STR profile (TD19-66). As little as a 2% population of fetal or maternal cells can be detected with this assay.

All source derived MSCs were incubated with one of the fluorescence-labeled antibodies: Pacific Blue anti-CD90.2, Pacific Blue anti-CD105, FITC anti-CD29, PE, FITC anti-CD79α, APC-Cy7 anti-CD45, PE anti-CD14, or PE anti-CD11. Cells were analyzed by flow-assisted cell sorting (FACS) Canto™ II (BD Biosciences; San Jose, CA). Isotype controls were used as negative controls. By FACS analysis, the isolated MSCs demonstrated the well characterized expression pattern of mesenchymal markers including positive expression of CD90.2⁺, CD105⁺, CD29⁺, Sca-1⁺, and lacked CD79α⁻, CD45⁻, CD14⁻, and CD11 expression (Table 2).

TABLE 2
FACS analysis of MSC cell markers (% positive)

| | | ASC | | Whole | Chorion | Villi |
|---|---|---|---|---|---|---|
| Antibody | POS/NEG | (Lonza) | WJ | cord | (CSC) | (CVC) |
| CD105 | ++++ | >90 | >90 | | 94 | 97 |
| CD90 | ++++/++ | >90 | >90 | >90 | 73 | 96 |
| CD73 | ++++ | >90 | >90 | >90 | 95 | 98 |
| CD11b | NEG | | 0 | | 1 | 0 |
| HLADR | NEG | | 0 | | 1 | 1 |
| CD45 | NEG | <5 | 0 | <10 | 0 | 0 |

### Example 4

### Evaluation of the Safety and Efficacy of Perinatal Stromal Cells Infusion on the Development of Fibrosis

To test the effect of WJ and whole cord MSCs against development of fibrosis, tail vein injection of cells was performed at day 1 post-bleomycin infusion.

Male C57BL/6 mice were obtained from the Jackson Laboratories (Bar Harbor, ME). 22-month old male mice were used for all experiments (n=6-10/group). 4-month old male C57BL/6 were used for isolation of ASCs. Animals were housed under pathogen-free conditions with food and water ad libitum. All experiments and procedures were approved by the Institutional Animal Care and Use Committee at University of Miami Miller School of Medicine (Miami, FL), a facility accredited by the American Association for the Accreditation of Laboratory Animal Care.

After induction of anesthesia with ketamine, bleomycin sulfate (Sigma-Aldrich Corp; St. Louis, MO) dissolved in 50 μl sterile saline was administered by direct intratracheal instillation (2.0 U/kg), to generate BLM-induced lung injury. Control mice received 50 μl of intratracheal sterile saline. Mice were weighed at baseline, day 7 post-BLM, and at sacrifice. Mice were sacrificed 21 days following BLM or saline administration. ASCs and all other source derived MSCs (passage 2 or 3) were thawed in a 37° C. water bath and washed in PBS to remove the cell freezing solution prior to injection. Cells were then passed through a 70 μm cell strainer to remove cell clumps. Cells were counted and resuspended in PBS immediately prior to injection. At 1 or 10 days post-BLM injury, mice were administered 5×10⁵ cells in 200 μl of PBS by tail vein injection over 1 minute. Control mice received 200 μl of PBS by tail-vein injection.

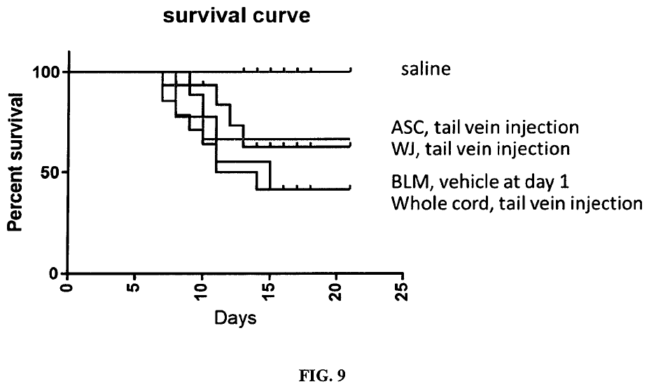

As illustrated in FIG. 9, and as previously reported, survival decreased after treatment with BLM. 50% of mice in the BLM group survived more than 14 days post-BLM administration. However, 80% of the mice in the ASC or 75% in the WJ group survived 21 days post-BLM administration. Mice receiving whole cord injections had ˜50% survival rate 21 days post-BLM.

To assess the effect of the tail vail injection of cells on the rescue from established fibrosis 10 days post-BLM infusion, Ashcroft scoring and collagen content were measured.

Pulmonary fibrosis was assessed by a pulmonary pathologist blinded to the experimental groups using the semi-quantitative Ashcroft method on Masson's Trichrome-stained slides at 20× magnification. Individual fields were assessed by systematically moving over a 32-square grid; each field was assessed for fibrosis severity and assigned a score on a scale of 0 (normal lung) to 8 (total fibrosis of the field) and an average was obtained for each slide. Collagen content was assessed by evaluating hydroxyproline content, which was determined according to the manufacturer's instructions (Hydroxyproline Assay Kit; Sigma-Aldrich, St. Louis, MO). Briefly, 2 mg lung fragments were weighed and homogenized in 100 μl of distilled water. An equal volume of 10 N HCl was added to the samples before drying at 49° C. for 3 hours. 50 μl of sample was loaded in the plate and incubated overnight at 37° C. A hydroxyproline standard curve was prepared according to a standard solution (between 0 and 1 μg/well). Hydroxyproline content was read at 557 nm, using the SoftMax Pro Software (Molecular Devices Corp; Sunnyvale, CA).

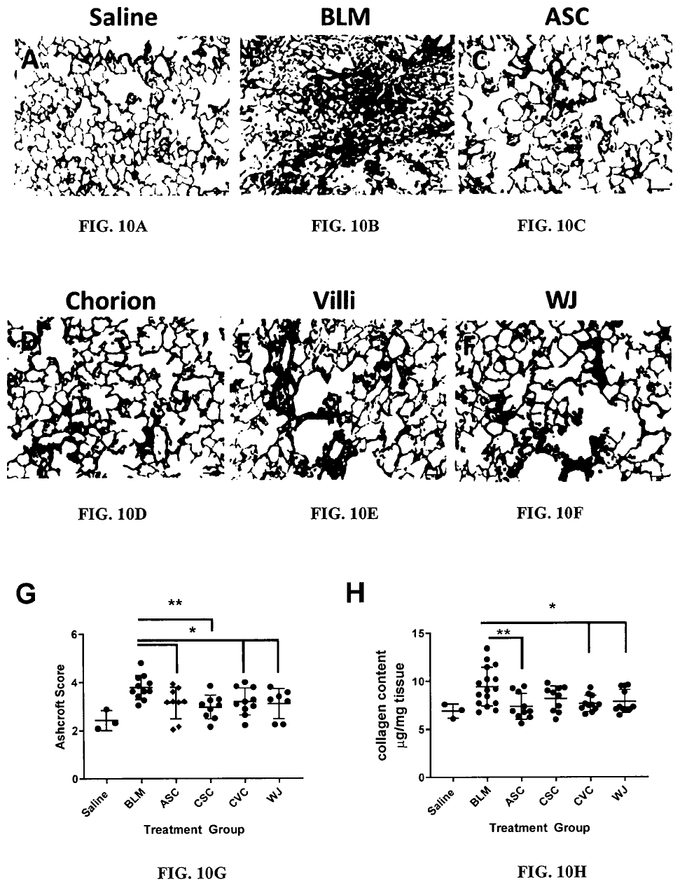

Due to clinical symptoms of cough and dyspnea that are nonspecific, most pulmonary fibrosis patients experience a delay in diagnosis and are diagnosed at later stages of the disease after significant fibrosis has occurred. It was found that at day 10, in this setting fibrosis was established, and the effect of the tail tail vein injection of MSCs could be evaluated. As shown in FIG. 10A-H all MSCs were found to reduce Ashcroft (FIG. 10G), and hydroxyproline measurements (FIG. 10H).

### Example 5

### Evaluation of the Effect of Perinatal Stromal Cells Infusion on Fibrosis and Inflammation

To assess the effect of the cells' infusion on molecular markers of fibrosis and inflammation, αᵥ-integrin, and TNFα levels of expression were measured by real-time polymerase chain reaction (RT-PCR). RNA were extracted from lung tissue. The TaqMan rRNA control reagents kit (Life Technologies) was used to detect 18S rRNA gene, an endogenous control, and samples were normalized to the 18S transcript content. For microRNA analyses, cDNA was generated using qScript™ microDNA cDNA Synthesis Kit (Quanta Biosciences, Beverly, MA) according to manufacturer's instructions. Amplification of microRNA-199-3p was performed using specific primers (IDT, Coralville, IA) using Real-Time SYBR Green qRT-PCR Amplication kit (Quanta Biosciences, Beverly, MA). U6 expression was used as a control for microRNA analyses, and relative expression was calculated using the comparative C(T) method.

As shown in Table 2, ASCs and WJs decreased mRNA expression of established molecular markers of fibrosis and inflammation. Administration of treatments (day 10 post-BLM) resulted in significant decrease in markers associated with BLM-induced pulmonary injury. Expression of αᵥ-integrin mRNA, a transmembrane cell adhesion molecule that modulates tissue fibrosis was found increased in BLM-treated mice compared to ASC, CVC and WJ derived cells (Table 3). TNF-α, a marker of inflammation, was also increased in BLM-treated mice compared to all treatments except CSC (Table 3).

TABLE 3
mRNA expression

| Cell type | αVintegrin | TNFα |
|---|---|---|
| BLM control (n = 14) | 1.8 ± 0.18 | 0.31 ± 0.07 |
| ASC (n = 15) | 0.71 ± 0.17*** | 0.03 ± 0.008** |
| Chorion (CSC, n = 11) | 1.2 ± 0.25 | 0.09 ± 0.012 |
| Villi (CVC, n = 9) | 1.19 ± 0.15* | 0.036 ± 0.008** |
| WJ (n = 11) | 0.5 ± 0.11*** | 0.05 ± 0.005* |

*P < 0.05,
**P < 0.001,
***P < 0.0001 compared to BLM control

Further, the effect of the cell infusion on fibrotic pathways activated by BLM administration was measured by evaluating AKT activation by western blot, and MMP-2 activity by zymography.

Western analysis was performed on homogenized lung tissue. For pAKT (Cell Signaling, 92715), AKT (Santa Cruz Biotechnology, Sc-1619), Caveolin 1 (Cell Signaling, 3267S) and β-actin (Sigma Aldrich, A5441). Five to twenty-five μg of protein lysate was fractionated on 10% polyacrylamide gels and transferred to nitrocellulose membranes. Immunoreactive bands were determined by exposing nitrocellulose blots to a chemiluminescence solution (Denville Scientific Inc.; Metuchen, NJ) followed by exposure to Amersham Hyperfilm ECL (GE Healthcare Limited; Buckinghamshire, UK). To determine the relative amounts of protein densitometry was evaluated using the Image J version 1.48 v (National Institutes of Health; Bethesda, MD). β-actin analysis was used as a control. Ratio of phosphorylated AKT to AKT protein expression in lung tissue of subjects was quantified by western analysis at 21-day sacrifice. Aged C57B1/6 mice treated with intratracheal BLM demonstrated increased pAKT/AKT protein expression compared to lungs from mice treated with tail vein injection of allogeneic ASCs or WJs 10 days following BLM administration. Inset shows a representative western blot and β-actin loading control.

Matrix metalloproteinase-2 (MMP-2) activity was measured on lung tissue. Briefly, samples and standards (Chemicon) were loaded onto 10% zymogram gels (Novex, ThermoFisher Scientific). Following electrophoresis, gels were incubated for 24 hours at 37° C. in a gelatinase solution to allow for determination of MMP-2 proteolytic activity without interference from associated tissue inhibitors. Relative MMP-2 activity was measured by densitometry using Image J version v1.48 (National Institutes of Health, Bethesda, MD). Aged C56B1/6 mouse lung expression of MMP-2 activity increased at 21-day sacrifice in response to bleomycin (BLM) lung injury. Treatment with allogeneic ASCs or WJs on day 10 post-BLM infusion resulted in consistent decreased MMP-2 activity compared to BLM-only control mice. Insert is representative zymogram.

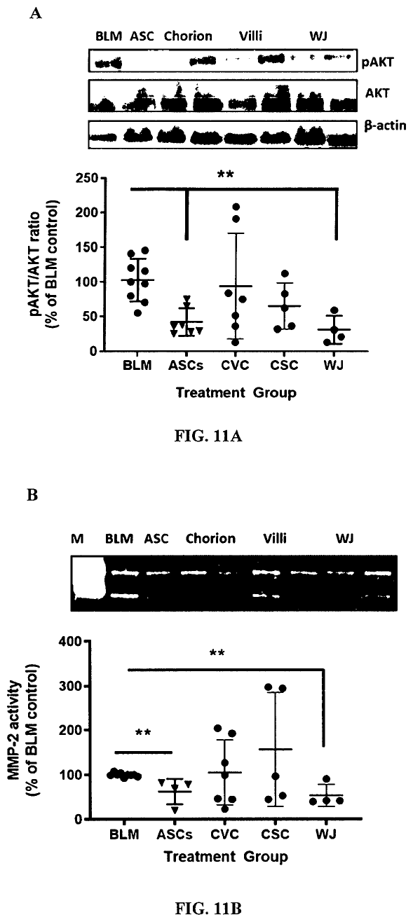

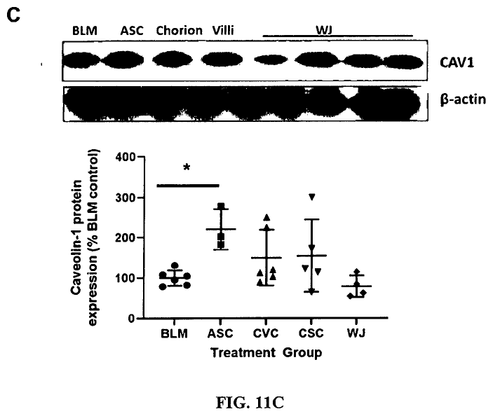

As illustrated in FIG. 11A-C, ASCs and WJ consistently suppressed AKT activation (FIG. 11A) and MMP-2 activity (FIG. 11B). Cav 1 protein, described as an anti-fibrotic, increased only with ASC treatment demonstrating their anti-fibrotic effect (FIG. 11C).

The effect of cells infusion on miR-29 and -199, which were shown to have importance in lung associated fibrotic pathways, were evaluated. As shown in Table 4, it was found that miR-199 expression was increased in BLM treated mice, and was decreased only by tail vein injection with ASCs. These data supported the regulation of Cav-1 only by ASCs.

TABLE 4
microRNA expression

| Cell type | MiR-199-3p/U6 relative expression |
|---|---|
| BLM control (n = 6) | 0.21 ± 0.04 |
| ASC (n = 7) | 0.07 ± 0.008* |
| Chorion (CSC, n = 5) | 0.11 ± 0.01 |
| Villi (CVC, n = 6) | 0.10 ± 0.02 |
| WJ (n = 6) | 0.16 ± 0.04 |

*P < 0.05 compared to BLM control

### Example 6

### Evaluation of the Immunoregulator Effect of Human Chorionic Villus Cells In Vitro

Chorionic villus cells from human placentas were isolated and used as cell therapeutic in models representative of chronic inflammation that leads to fibrosis using three different but complementary animal models.

As illustrated in FIG. 12A-B, human stromal cells were isolated from CD163+, CD11b−, PanCK+, PDL1+ chorionic villus tissue (FIG. 12B) and had fibroblast like morphology, were plastic adherent, and had a phenotype of CD105+, CD90+, CD73+, CD273+, CD210+, CD178−, CD119−, CD85d−, CD40−, CD11b−, HLADR−, CD45− (FIG. 12A).

Mature monocyte-derived dendritic cells (moDC) are cells from the innate immune system with antigen presenting capabilities (Antigen Presenting Cells) that can stimulate “inflammation” by activating T cells. Mature moDC can be measured by the upregulation of maturity markers such as CD1a, CD83. In contrast, immature moDC have properties of tolerogenic dendritic cells, which inhibits “inflammation” by disabling T cell proliferation, immature moDCs can be measured by the downregulation of mature markers such as CD1a, CD83 and by the simultaneous upregulation of markers of tolerogenesis such as CD85d, and CD14.

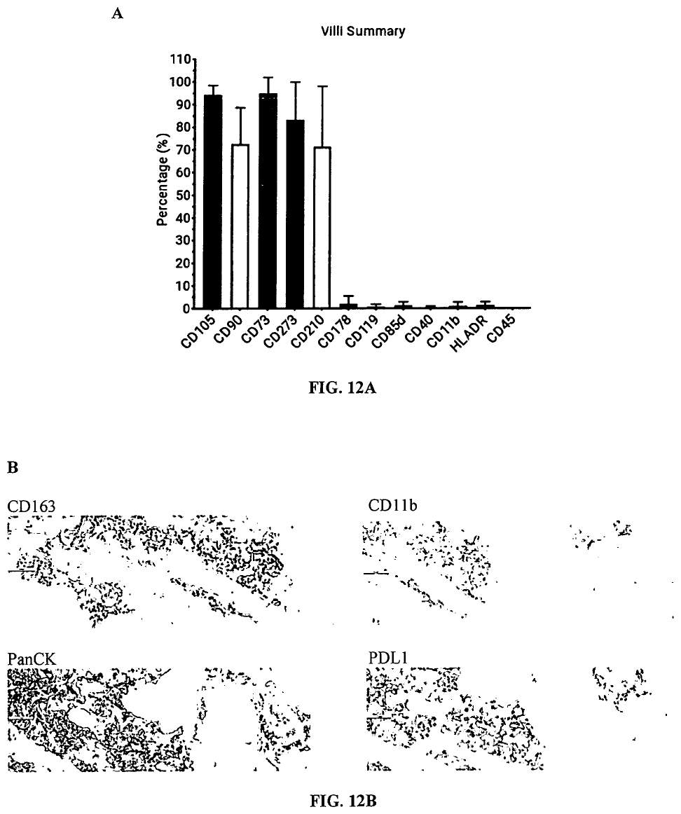

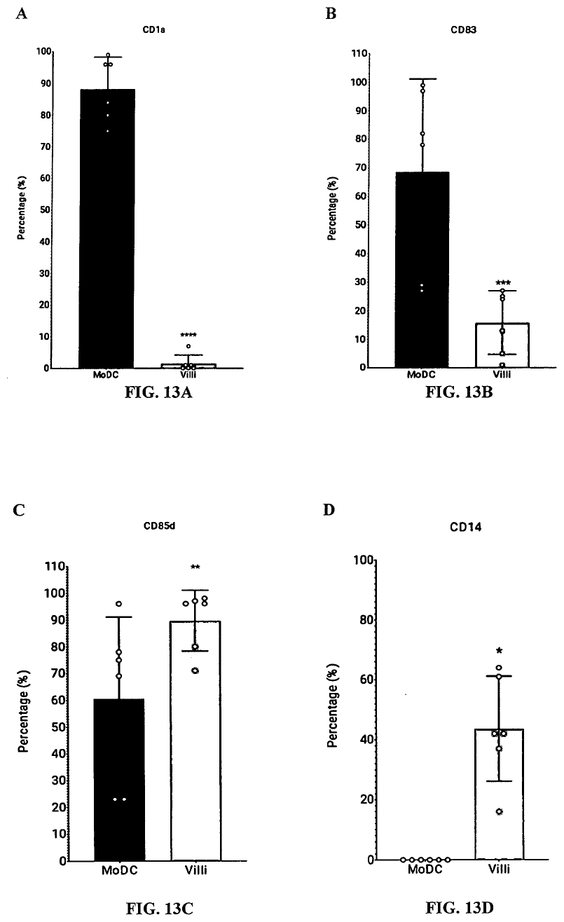

To assess the immune modulating abilities of the villi stromal cells, the expression of maturity markers CD1a and CD83, CD85d, and CD14, were measured. As illustrated in FIG. 13A-D, it was found that mature markers of moDC were significantly downregulated in the presence of villi stromal cells villi stromal cells (FIGS. 13A and 13B) and that tolerogenic markers of moDC were significantly upregulated in the presence of villi stromal cells (FIGS. 13C and 13D). Villi stromal cells were capable of immune modulating both adaptive immune cells and innate immune cells, representative of both major compartments of the entire immune system.

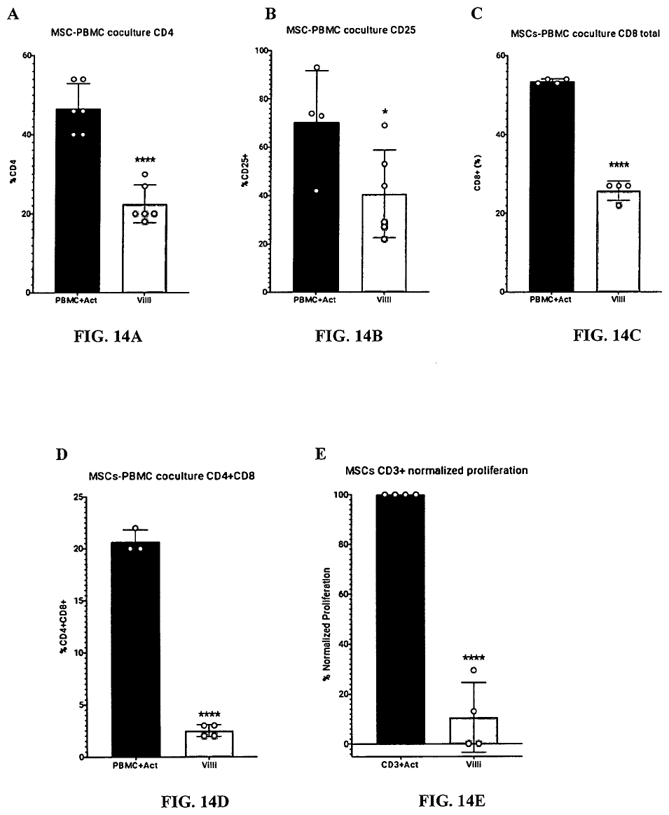

Further, the immune-suppressive effect of villi stromal cells was evaluated on various cell population of peripheral blood mononucleated cells (PBMC) allostimulated with CD3/CD28/CD2. As illustrated in FIG. 14, villi stromal cells were found to have a significant immune-suppressive effect on CD4+, CD25+, CD8+, CD4+ and CD8+, and CD3+ proliferation.

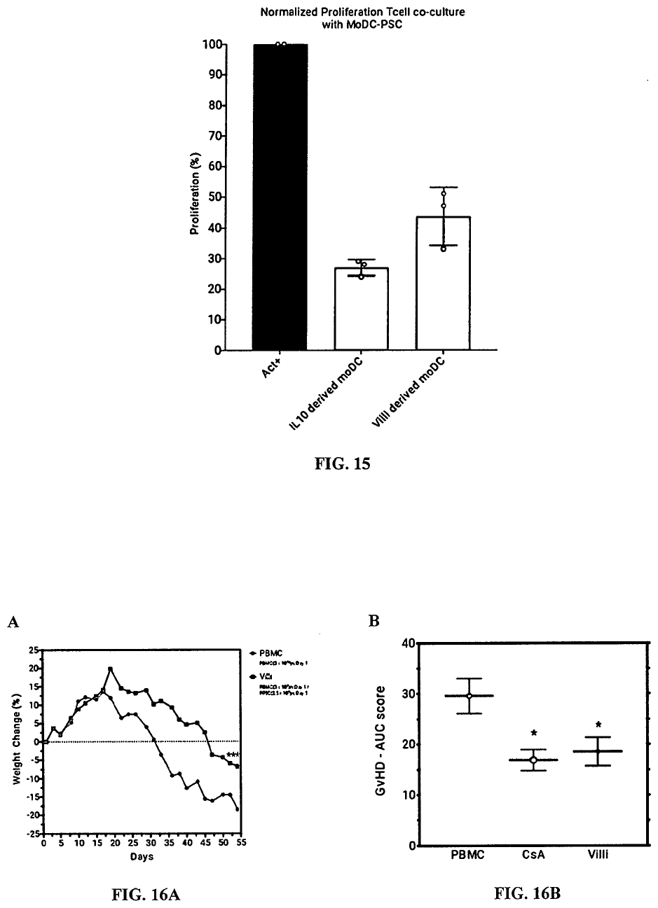

The effect of villi stromal cells on human immune tolerance was also evaluated, by measuring the changes in the proliferation of T cells, when co-cultured with various moDCs. IL10 derived moDC were dendritic cells derived from allogeneic monocytes in the presence of IL10, which has been determined as a positive control for a tolerogenic dendritic cells. Villi derived moDC cells, were allogeneic monocyte derived dendritic cells in the presence of villi cells; cells were cultured in separate compartments using a well insert, and therefore the paracrine effect of the villi cells was observed. As shown in FIG. 15, the tolerogenic IL10-moDCs were not capable of promoting complete T cell proliferation/stimulation (as compared to the maximal proliferation of T cell obtained using an allo-antigen CD3/CD2/CD28), defining them as tolerogenic by nature; and villi cells were found to induce a significant promotion of human immune tolerance, by promoting tolerogenic dendritic cells that showed tolerance characteristics on human T cells.

### Example 6

### Evaluation of Effect of Human Chorionic Villus Cells in a in Vivo Humanized Graft Versus Host Disease Model

To assess the effect of human chorionic villus stromal cells in vivo, the cells were administered to mice, in a Humanized Graft Versus Host Disease Model, Xenogeneic GVHD NSG (Non-obese diabetic/severe combined immunodeficient-IL-2 receptor gamma-null (NOD.Cg-Prkd^{scid} Il2rg^{tm1wjl}/SzJ, NSG®). GVHD was generated in female NSG mice, which were engrafted intravenously (IV) via tail vein injection with 3×10⁷ human PBMCs. A positive control (PBMC) group received no treatment which allowed for GvHD development. A negative control (CsA) group was administered with Cyclosporin A once daily intraperitoneally (IP) at 15 mg/kg to study end (qd to end) from Day 1 to repress GvHD symptoms. On Day 5, mice received MSC cell infusions (Villi) as a single dose of 350,000 of cells, delivered by intravenous tail vein injection.

Animal survival was monitored daily, weight and GvHD scoring were assessed three times a week. Clinical GVHD index was scored based on 5 parameters: weight loss, activity, posture, fur texture, and skin integrity; on a scale of 0 to 2, with 2 being most severe. Animals were euthanized when their weight reached a weight loss of >30% or two consecutive loss measurements of >25%. Five animals in each group were analyzed at day 40 for expression of human CD3+ in whole blood (0.1 mL was collected by mandibular bleeds). Experiment was carried out until day 55. As illustrated in FIG. 16, weight loss which as representative of the progression of the GvHD disease, was significantly reduce by the administration of Villi stromal cells and the score (AUC) of the disease progression was also positively impacted.

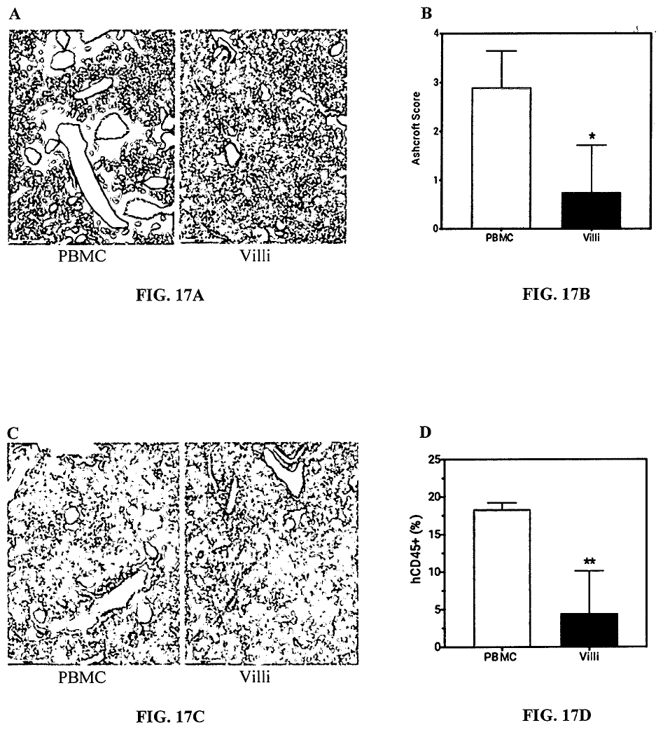

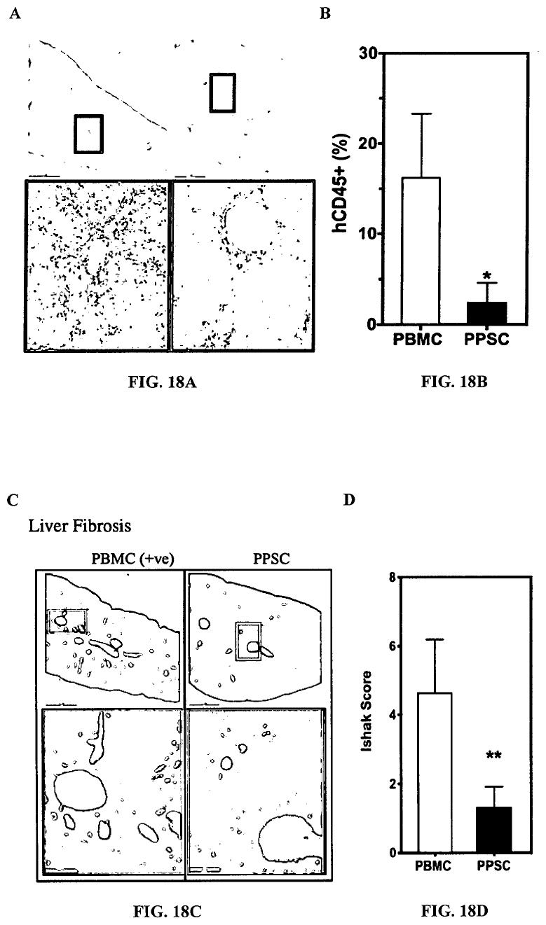

As shown in FIGS. 17 and 18, histological evaluations demonstrated the decrease in pulmonary and liver fibrosis, Ashcroft and Ishak, respectively; in addition to a significant reduction in human immune (CD45+) cell infiltration into the organs, responsible for inflammatory insults.

### Example 6

### Evaluation of Effect of Human Chorionic Villus Cells in a in Vivo Model of Dilated Cardiomyopathy

To assess the capability of Villi stromal cells to reduce cardiac fibrosis human chorionic villus cells were administered to mice, in an auto-immune mice model of dilated cardiomyopathy (DCM). Experimental autoimmune myocarditis (EAM) is a widely-accepted animal model of myocarditis and can be induced by active immunization of susceptible mouse strains with myosin antigens or adoptive transfer of myosin-reactive T lymphocytes. Disease peaks 2-3 weeks after immunization, is often more severe in male mice, and is characterized by extensive infiltration of T cells and APCs into the myocardium. A critical role for IL-17 produced by myosin-reactive Th17 cells has been described. Importantly, most mice with EAM progress to DCMi by approximately 6-8 weeks after immunization, characterized by cardiac fibrosis and myocardial remodeling, thereby replicating the signature features of human DCMi.

To evaluate whether progression of EAM to DCMi in mice can be ameliorated or prevented by treatment with villi stromal cells, villi stromal cells have been administered to a mice model of EAM. EAM and DCMi were induced in Wt BALB/c mice by immunization with cardiac myosin peptide (MyHCα₆₁₄₋₆₂₉). On day 11 after immunization mice were treated with Villi stromal cell preparations. DCMi was scored on day 42 by assessing collagen deposition (Fibrosis) as determined by Massons Trichrome staining. On day 42 spleen was removed and single cell suspensions prepared for cytokine ELISPOT assay. Frequencies of IFN-γ producing T cells and frequencies of IL-17 producing T cells were evaluated.

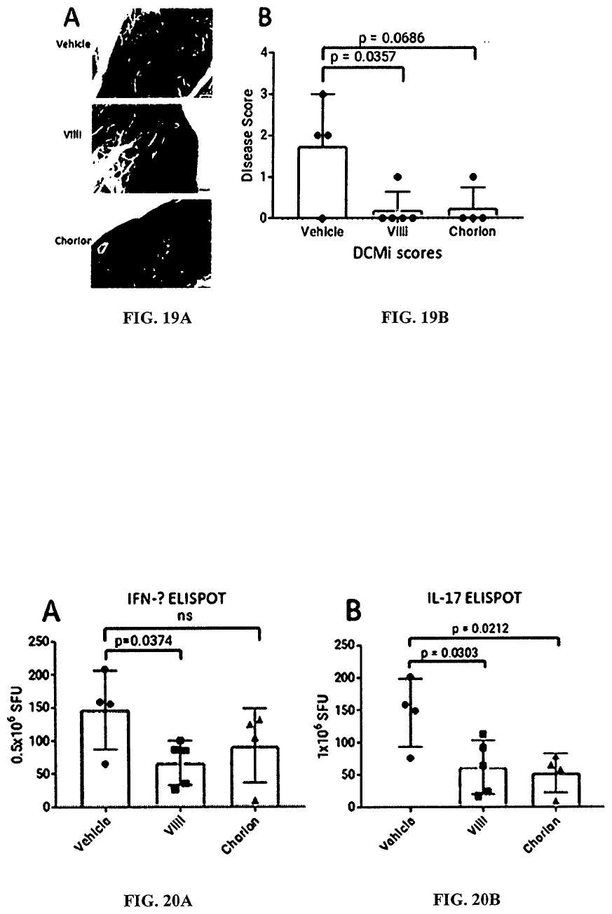

As illustrated in FIG. 19, villi stromal cell treatment of mice with EAM was found highly effective in preventing the progression towards DCMi and was capable of reducing fibrosis. Further, and as illustrated in FIG. 20, villi stromal cells treatment significantly decreased the production of T cell-derived pathogenic cytokines in mice with EAM. The results suggest that villi MSCs may be a novel treatment modality for DCMi and may change current treatment paradigms, since it was capable of reducing fibrotic scores in heart and downregulate proinflammatory cytokines such as INFg and IL17.

### Example 7

### Evaluation of Effect of Human Chorionic Villus Cells in a in Vivo Model of Induced Pulmonary Fibrosis

To assess the capabilities of Villi stromal cells to reduce pulmonary fibrosis, villi stromal cells were administered in a chemically induced model of pulmonary fibrosis in mice. For this model, Male C57BL/6 mice were treated with bleomycin sulfate (BLM) by direct intratracheal instillation and sacrificed 21 days following BLM.

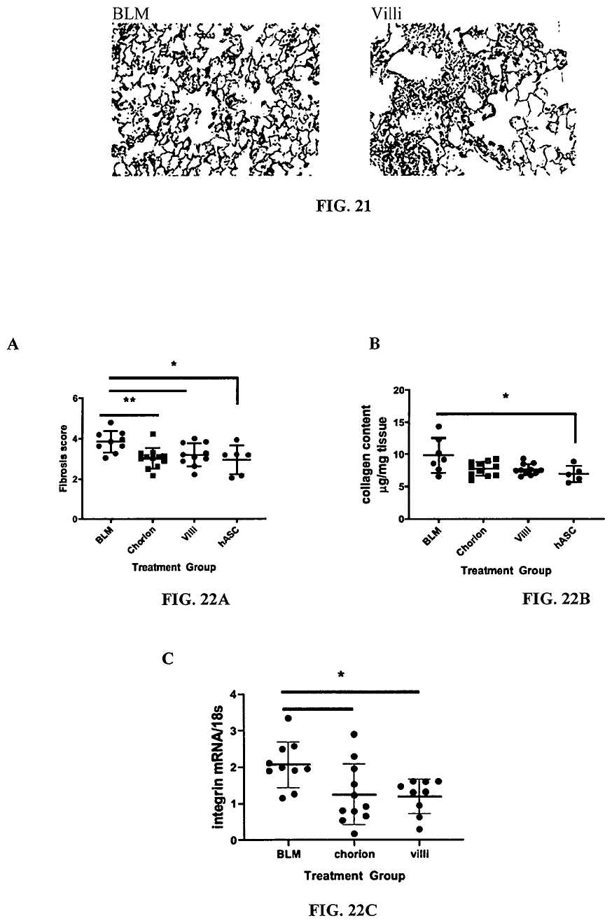

As illustrated in FIG. 21 and FIG. 22, collagen deposition was significantly reduced by infusion of villi stromal cells and consequently fibrosis score was significantly reduced. In addition, Villi stromal cells decreased inflammation and downregulated integrin, which mediates fibrosis.

Although the invention has been described with reference to the above examples, it will be understood that modifications and variations are encompassed within the spirit and scope of the invention. Accordingly, the invention is limited only by the following claims.

What is claimed is:

1. A perinatal stromal cell (PSC) composition comprising at least one of the following cell types: (i) amnion perinatal stromal cells (APSCs), (ii) placenta proper stromal cells (PPSCs), (iii) Wharton's jelly perinatal stromal cells (WPSCs), (iv) whole chorion derived stromal cells (CSCs), and (v) chorionic villi-derived stromal cells (CVCs), and a pharmaceutically acceptable carrier.

2. The PSC composition of claim 1, which comprises at least two of the following cell types: (i) APSCs, (ii) PPSCs, (iii) WPSCs, (iv) CSCs, and (v) CVCs.

3. The PSC composition of claim 1, which comprises at least three of the following cell types: (i) APSCs, (ii) PPSCs, (iii) WPSCs, (iv) CSCs, and (v) CVCs.

4. The PSC composition of claim 1, which comprises at least four of the following cell types: (i) APSCs, (ii) PPSCs, (iii) WPSCs, (iv) CSCs, and (v) CVCs.

5. The PSC composition of claim 1, which comprises all five of the following cell types: (i) APSCs, (ii) PPSCs, (iii) WPSCs, (iv) CSCs, and (v) CVCs.

6. The PSC composition of any one of the foregoing claims, wherein at least one of the APSCs, PPSCs, WPSC, or CVCs express a molecular marker selected from the group consisting of CD105, CD90, CD73, CD273, CD210, and a combination thereof.

7. The PSC composition of any one of the foregoing claims, wherein at least one of the APSCs, PPSCs, WPSCs, or CVCs does not express a molecular marker selected from the group consisting of CD11b, CD45, HLADR, CD119, CD85b, CD178, CD40, and a combination thereof.

8. The PSC composition of any one of the foregoing claims, wherein said cells are derived from more than one donor.

9. The PSC composition of any one of the foregoing claims, wherein said cells are derived from more than one donor of the same blood type.

10. The PSC composition of any one of the foregoing claims, wherein said cells are derived from more than one donor which have been determined to be histocompatible with each other.

11. The PSC composition of any one of the foregoing claims, wherein said cells are derived from more than one donor which have been determined to comprise the same or comprise similar human leukocyte antigens (HLA) alleles or major histocompatibility complex (MHC).

12. The PSC composition of any one of the foregoing claims, wherein said cells are derived from one or more donors wherein the DNA thereof has been analyzed to confirm that the cells do not comprise gene mutations including those correlated to genetic diseases including but not limited to cancer, Autosomal dominant diseases such as familial hypercholesterolemia, Neurofibromatosis type I, Hereditary spherocytosis, Marfan syndrome, Huntington's disease, Autosomal recessive diseases such as Sickle cell anemia, Cystic fibrosis, Tay-Sachs disease, Phenylketonuria, Autosomal recessive polycystic kidney disease, Mucopolysaccharidoses, Lysosomal acid lipase deficiency, Glycogen storage diseases such as Galactosemia, X-linked diseases such as Duchenne muscular dystrophy, and Hemophilia.

13. The PSC composition of any one of the foregoing claims, wherein said cells are derived from one or more donors which have been determined not to comprise any pathogenic viruses or other microbial pathogens.

14. A method of reducing chronic inflammation and consequent tissue fibrosis in a subject comprising administering to the subject in need thereof the perinatal stromal cell (PSC) composition of any one of the foregoing claims.

15. A method of treating or preventing fibrosis in a subject in need thereof comprising administering to the subject in need thereof the perinatal stromal cell (PSC) composition of any one of the foregoing claims.

16. The method of claim 14 or 15, wherein the subject comprises one or more of lung or pulmonary fibrosis, e.g., caused by infection, chemotherapy, cancer, COPD, environmental insults such as asbestos, coal dust and the like, or a disease such as cancer or cystic fibrosis; liver fibrosis e.g., caused by alcoholism, fatty liver disease, nonalcoholic steatohepatitis (NASH), Nonalcoholic fatty liver disease (NAFLD), hepatitis B or hepatitis C; heart fibrosis e.g., caused by disease, infection, heart attack or stroke; Mediastinal fibrosis characterized by calcified fibrosis of the lymph nodes; retroperitoneal cavity fibrosis; bone marrow fibrosis; skin fibrosis; or scleroderma or systemic sclerosis.

17. The method of any one of claims 14-16, wherein the fibrotic tissue comprises a lung, liver, heart, pancreas, blood vessel, large intestine, small intestine, kidney, skin, interstitium, or a scar tissue.

18. The method of any one of claims 14-17, wherein a collagen content and or a collagen deposit in the tissue is reduced as compared to the collagen content or collagen deposit in said tissue before the administration of the PSC composition.

19. The method of any one of claims 14-18, wherein a fibrotic score in the tissue is decreased as compared to said fibrotic score in the tissue before the administration of the PSC composition.

20. The method of claim 19, wherein the fibrotic score is selected from the group consisting of an Ashcroft score and an Ishak score.

21. The method of any one of claims 14-20, wherein a molecular marker of fibrosis is decreased in the tissue as compared to said molecular marker in the tissue before the administration of the PSC composition.

22. The method of claim 21, wherein the molecular marker of fibrosis is selected from the group consisting of αᵥ-integrin expression, MMP-2 activity, pAKT/AKT expression ratio, miR199 expression, and a combination thereof.

23. The method of any one of claims 14-22, wherein an anti-fibrotic molecular marker is increased in the tissue as compared to said molecular marker in the tissue before the administration of the PSC composition.

24. The method of claim 23, wherein the anti-fibrotic molecular marker is Caveolin-1 expression.

25. A method of reducing or preventing tissue or organ inflammation in a subject in need thereof comprising administering to the subject in need thereof the perinatal stromal cell (PSC) composition of any one of claims 1-13.

26. The method of any one of claims 14-25, wherein a molecular marker of inflammation in a tissue is decreased as compared to said molecular marker in the tissue before the administration of the PSC composition.

27. The method of claim 26, wherein the molecular marker of inflammation is selected from the group consisting of TNFα expression, INFγ expression, IL-17 expression, and a combination thereof.

28. The method of any one of claims 14-27, wherein CD45+ T cell infiltration in a tissue is decreased as compared to before the administration of the PSC composition.

29. A method of inducing immune tolerance in a subject comprising administering to the subject in need thereof the perinatal stromal cell (PSC) composition of any one of claims 1-13.

30. The method of claim 29, wherein the proliferation of pro-inflammatory mature monocyte-derived dendritic cells (moDC) is inhibited, the proliferation of tolerogenic immature moDC is induced, the proliferation of pro-inflammatory CD4+, CD8+, CD3+, CD4+CD8+ (double positive), and/or CD25+ T cells is inhibited, and/or the proliferation of CD11b+, CD11c+ T cells is inhibited.

31. The method of claim 29 or 30, wherein a decreased expression of maturity markers CD1a and CD83 in a monocyte population indicates an inhibition of mature moDC proliferation, and wherein an increased expression of immaturity markers CD85d and CD14 in a monocyte population indicates an increase of immature moDC proliferation.

32. The method of any one of claims 14-31, wherein the subject has single or multi-organ fibrosis, idiopathic pulmonary fibrosis (IPF), interstitial lung disease (ILD), dilated cardiomyopathy (DCM), or graft versus host disease (GVHD).

33. A method of treating single- or multi-organ fibrosis, idiopathic pulmonary fibrosis (IPF), interstitial lung disease (ILD), dilated cardiomyopathy (DCM), or graft versus host disease in a subject (GVHD) comprising administering to the subject in need thereof the perinatal stromal cell (PSC) composition of any one of claims 1-13.

34. The method of any one of claims 14-33, wherein the PSC composition comprises chorionic villi-derived stromal cells (CVC), and a pharmaceutically acceptable carrier.

35. The method of any one of claims 14-33, wherein the subject has or is at risk of developing acute respiratory distress syndrome (ARDS) or sepsis.

36. The method of any one of claims 14-35, wherein the subject has an acute or chronic viral disease or infection, e.g., hepatitis A, B, C, D or E, influenza, herpes, HIV, encephalitis, dengue, Human Metapneumovirus (HMPV), Arthritogenic alphavirus, respiratory syncytial virus (RSV) or coronavirus infection such as SARS-CoV, SARS-CoV-2, MERS, and/or has an acute or chronic bacterial disease or infection, e.g., influenza or pneumococcal infection, optionally one that puts the subject at risk of developing acute respiratory distress syndrome (ARDS) or sepsis or fibrosis.

37. The method of any one of claims 14-36, wherein the subject has tissue or organ inflammation, e.g., pericarditis.

38. The method of claim 37, wherein said tissue or organ inflammation is caused by a vaccine, optionally an mRNA vaccine or by a rejection response against a transplanted tissue or organ or cell therapy.
